(19) United States
(12) Patent Application Publication
(10) Pub. No.: US 2018/0035983 A1
(43) Pub. Date: Feb. 8, 2018

(54) CELL AND TISSUE COLLECTION METHOD AND DEVICE

(71) Applicant: Histologics LLC, Anaheim, CA (US)

(72) Inventor: Neal Marc Lonky, Yorba Linda, CA (US)

(73) Assignee: Histologics LLC, Anaheim, CA (US)

(21) Appl. No.: 15/709,790

(22) Filed: Sep. 20, 2017

Related U.S. Application Data
(63) Continuation of application No. 13/830,657, filed on Mar. 14, 2013.
(60) Provisional application No. 61/621,377, filed on Apr. 6, 2012.

(51) Int. Cl. A61B 10/02

(52) U.S. Cl. CPC A61B 10/02; A61B 2010/0216

(57) ABSTRACT

In an embodiment of the invention, a frictional tissue sampling device with an expandable balloon and associated abrasive material can be used to obtain tissue biopsy samples. A frictional tissue sampling device with expandable abrasive material can be used to obtain an epithelial tissue biopsy sample from lesions. The device can be otherwise used to sample specific locations. In various embodiments, the head of the device can be passes through a catheter into a body canal to sample epithelial tissue.

### PRIORITY CLAIM

This application is a continuation of and claims priority to (1) U.S. Utility application Ser. No. 13/830,657 entitled “CELL AND TISSUE COLLECTION METHOD AND DEVICE” filed Mar. 14, 2013 which claims priority to (2) the U.S. Provisional Application No. 61/621,377, entitled “CELL AND TISSUE COLLECTION METHOD AND DEVICE” by Neal M. Lonky filed Apr. 6, 2012. These applications (1)-(2) are herein expressly incorporated by reference in their entireties.

### FIELD OF THE INVENTION

The invention relates to epithelial tissue sampling and collection devices for analysis including performing biopsies.

### BACKGROUND OF THE INVENTION

Lesions represent one type of area that is frequently targeted by tissue sampling and collection devices. A lesion is caused by any process that alters or damages tissue. A lesion can be defined as any pathological, morphological, or traumatic discontinuity of tissue with partial loss of tissue function. The concept of a lesion includes wounds, sores, ulcers, tumors, cataracts and any other tissue damage. Lesions can range from the skin sores associated with eczema to the changes in lung tissue that occur in tuberculosis. Generally, a lesion can be characterized by the epithelium covering the connective tissue becoming exophytic, endophytic, discolored, hypertrophic or atrophic, and fragile, leading to ulceration and bleeding. Some causes of lesions and other epithelial abnormalities are described below.

Human papillomaviruses (HPV) are responsible for many cutaneous and mucosal lesions. Some viral genotypes are considered to be the causal agents of cervical cancer. Natural genital HPV infection seems to be poorly immunogenic because of its nonproductive and non-inflammatory characteristics and also because of mechanisms developed by the virus to counteract the immune response.

Cervicovaginitis refers to inflammation of the squamous epithelium of the vagina and cervix caused by an inflammatory reaction to an infection. This damage leads to desquamation and ulceration, which can cause a reduction in the epithelial thickness due to loss of superficial and part of the intermediate layers of cells. In the deeper layers, the cells are swollen with infiltration of neutrophils in the intercellular space. The surface of the epithelium is covered by cellular debris and inflammatory mucopurulent secretions. The underlying connective tissue is congested with dilatation of the superficial vessels and with enlarged and dilated stromal papillae. Rare and uncommon cervical infections, due to tuberculosis, schistosomiasis and amoebiasis, cause extensive ulceration and necrosis of the cervix with symptoms and signs mimicking invasive cancer. Herpes simplex virus (HSV) can be present on the mucosal lining of the mouth or genitals. A large coalesced ulcer due to HSV can also mimic the appearance of invasive cancer. Chronic inflammation causing recurrent ulceration and healing of the cervix can result in a distortion of the cervix. Infections with the pathogenic fungi Cryptococcus neoformans, Histoplasma capsulatum, and Coccidioides immitis can be disseminated and some, e.g., C. neoformans, can result in pneumonia or meningitis. Longstanding viral, bacterial, fungal or protozoal infection and inflammation can lead to white or pink appearance as a result of fibrosis.

A biopsy can resolve the causative agent in many if not all of the lesions that are formed from viral, bacterial, fungal or protozoal infections. In the case of HSV, the sample must include cells, not just fluid from the blister, since the virus is in the skin cells of the blister or ulcer. The sample from a lesion or blister collected during an acute outbreak can be used to identify the agent based on the growth of the infectious substances and conventional or proteomics based analysis and detection. Epithelial abnormalities which can require cytological or histologic examinations can occur on the external body surface or internal body cavity. The target tissues for trans-epithelial sampling are likely to be sites where neoplasm is suspected such as skin moles or nevi, keratotic lesions, warts, ulcers, leukoplakic or otherwise discolored lesions, or exophytic lesions. Other situations where an epithelial tissue sampling and collection device can be used to collect DNA. Currently, a buccal or oral swab can be used to collect DNA samples from individuals. Dermal tape or abrasives can be used to collect DNA samples from superficial skin for clinical testing or forensic examinations

Previous epithelial tissue sampling and collection devices include brushes with rigid bristles that puncture and shear epithelial surfaces (U.S. Pat. Nos. 5,535,756; 6,258,044; 6,376,905; 6,494,845 and 6,132,421), single metal or plastic curettes that extend in a parallel direction to the applicator handle and are much larger than the innovation (U.S. Pat. Nos. 4,641,662 and 6,730,085), scalpels or similar bladed sharp cutting tools (U.S. Pat. Nos. 5,857,982; 5,800,362; 3,774,590; 5,092,345; 4,061,146; 5,868,668; 6,053,877; 5,470,308; 7,137,956, 4,168,698 and 4,757,826; and U.S. Publication Nos. 2005/0059905 and 2007/0093727), or very large electrified metal loops used to produce excisional biopsies (U.S. Pat. Nos. 5,913,857 and 5,951,550). One device performs simultaneous brush cytology and scrape biopsy on structures with an organic duct (U.S. Pat. No. 5,535,756). U.S. Pat. No. 5,643,307 “Colposcopic Biopsy Punch with Removable Multiple Sample Basket” has also been proposed to obtain biopsy samples when examining the cervix.

### SUMMARY OF THE INVENTION

In an embodiment of the present invention, a biopsy device includes a head with a rough or sharp edge and abrasive material applied to the surface of the head. Rotation of the head of the biopsy device on a site causes the rough or sharp edge and the abrasive material to dislodge epithelial tissue extending three layers deep to sample the striatum corneum through frictional tissue disruption or derma-abrasion. In an embodiment of the present invention, the rough or sharp edge dislodges cells which can be captured in the abrasive material together with additional cells dislodged by the abrasive material such that the frictional tissue disruption or derma-abrasion can be used for collecting samples for molecular testing. In an embodiment of the present invention, the head and the abrasive material can be ultrasonically welded together. In an alternative embodiment of the present invention, the abrasive material is attached to the device using an adhesive. In various embodiments of the present invention, an ultra violet (UV) light activated adhesive can be used to affix the abrasive material to the device. A railing or dam can be introduced onto the facet of the head of the device and the UV light activated adhesive is placed within the confines of the dam made on the facet by the railing.

In an embodiment of the present invention, a radial balloon with an abrasive material affixed to the exterior of the balloon allows the balloon to be passed through a body cavity in a deflated condition and inflated at a chosen site in order to functionally abrade the epithelial surfaces of the body cavity at the site. In an embodiment of the present invention, the balloon can be inserted via a catheter and expanded to fill the body cavity including a canal so that the fabric abuts the cavity epithelial surface. In an embodiment, the deflated balloon with its affixed abrasive material is recessed within a catheter or sheath until a lesion site or anatomical landmark is reached. The balloon can be extruded from the sheath or the sheath withdrawn exposing the balloon which can be inflated allowing contact with the anatomical area or lesion for the purposes of biopsy or sampling. In an embodiment of the present invention, a rigid, semi-rigid, or flexible catheter leading to the balloon can be manipulated to move the balloon inside or outside the sheath at a particular location. In an embodiment of the present invention, the balloon can be inflated through a lumen within a catheter with little or no resistance. In an embodiment of the present invention, the balloon once inflated can be rotated by applying a force that is exerted on a cannula or other device attached to the balloon which is accessible where the cannula exits the catheter. In an embodiment of the present invention, the position of the balloon once inflated can be adjusted by applying a force that is exerted on a cannula or other device attached to the balloon which is accessible where the cannula exits the catheter. In an embodiment of the present invention, the abrasive material is affixed to the exterior of the balloon in a single radial position. In an embodiment of the present invention, the abrasive material is affixed to the exterior of the balloon in a plurality of radial positions. In an embodiment of the present invention, rotation of the balloon causes the abrasive material to abrade the epithelial tissue at the surface of the body cavity attaching cellular tissue from the chosen site onto the abrasive material. The balloon can then be deflated and withdrawn from the body cavity to obtain the cell tissue sample. In an embodiment of the present invention, the abrasive material includes having sufficient flexibility and rigidity to frictionally abrade the epithelial surfaces. In an embodiment of the invention, the balloon can be re-sheathed prior to removal from the body cavity to cover and protect the abrasive surface thus facilitating maximal sample or tissue retention, and avoiding spillage of such tissue samples from inadvertent contact with the remaining tissue walls or other adjacent instrumentation. In an embodiment of the invention, the edge of the sheath is rough or sharp and can be used to dislodge cells which are then captured by the abrasive material affixed to the exterior of the balloon.

In an embodiment of the present invention, a retractable brush can be passed through a sheath where the sheath enters a body cavity and the brush can be ejected from the sheath at a chosen site in order to functionally abrade the epithelial surfaces of the body cavity at the site. In an embodiment of the present invention, a retractable brush comprised of abrasive material can be directly inserted and withdrawn into a body cavity or surface, or passed through a sheath where the sheath enters the body cavity. The brush with such abrasive material can be ejected from the sheath at a chosen site to functionally abrade the epithelial surfaces of the body cavity at that site. A hybrid device comprised of abrasive material at its midsection and base and retractable straight bristle brush can be used to sample surface and body cavity structures.

### BRIEF DESCRIPTION OF THE DRAWINGS

This invention is described with respect to specific embodiments thereof. Additional features can be appreciated from the Figures in which:

FIG. 1A is a cross section perspective of a device for insertion into a body cavity according to an embodiment of the invention;

FIG. 1B is a cross section perspective of the device of FIG. 1A after inflation of the balloon places the abrasive material attached to the balloon in contact with epithelial tissue from the surface of the body cavity and showing that rotation (clockwise) of the device, according to an embodiment of the invention;

FIG. 1C is a frontal view of the device shown in FIG. 1B for collecting epithelial tissue from the surface of a body cavity according to an embodiment of the invention;

FIG. 2A is a cross section perspective of a balloon device for insertion into a body cavity according to an embodiment of the invention;

FIG. 2B is a cross section perspective of the device of FIG. 2A with the balloon partially inflated and the abrasive material attached to the balloon being raised out of the device, according to an embodiment of the invention;

FIG. 2C is a cross section perspective of the device of FIG. 2B after inflation of the balloon places the abrasive material attached to the balloon in contact with epithelial tissue on the surface of the body cavity and showing that rotation (clockwise) of the device, according to an embodiment of the invention;

FIG. 2D is a cross section perspective of the device of FIG. 2C being deflated and the abrasive material attached to the balloon being lowered into the device, according to an embodiment of the invention;

FIG. 2E is an overhead perspective of the device of FIG. 2A showing an oval opening in the cannula from which the balloon can exit and/or reenter, according to an embodiment of the invention;

FIG. 3A is a cross section perspective of a brush in a sheath for insertion into a body cavity according to an embodiment of the invention;

FIG. 3B is a cross section perspective of the brush of FIG. 3A after ejection out of the sheath showing the bristles of the brush for contacting the epithelial tissue from the surface of the body cavity, according to an embodiment of the invention;

FIG. 4 is a cross section perspective of a hybrid balloon and brush after partial ejection out of the sheath showing the abrasive material attached to the balloon and the bristles of the brush attached to the cannula for contact with epithelial tissue from the surface of the body cavity, according to an embodiment of the invention;

FIG. 5A is a cross section perspective of a device with two surfaces for sampling two areas of the cervix, according to an embodiment of the invention;

FIG. 5B is a cross section perspective of one of the two surfaces shown in the device of FIG. 5A, according to an embodiment of the invention;

FIG. 5C is a frontal view of the surface shown in FIG. 5B for collecting epithelial tissue from one of the two areas of the cervix and showing that rotation (clockwise) of the device, according to an embodiment of the invention;

FIG. 6A is a cross section perspective of a device in a catheter or sheath with two surfaces for sampling two areas of the cervix, according to an embodiment of the invention;

FIG. 6B is a cross section perspective of the device of FIG. 6A after ejection out of the sheath showing the two surfaces in a convex arrangement for contacting the epithelial tissue from the cervix, according to an embodiment of the invention;

FIG. 6C is a cross section perspective of the device of FIG. 6A after ejection out of the sheath showing the two surfaces in a concave arrangement for contacting the epithelial tissue from the cervix, according to an embodiment of the invention;

FIG. 7A is a cross section perspective of a device for sampling the cervix, according to an embodiment of the invention;

FIG. 7B is a cross section of an arm of the device of FIG. 7A, according to an embodiment of the invention;

FIG. 7C is a cross section perspective of the device of FIG. 7A inserted into the cervix showing the direction of rotation, according to an embodiment of the invention;

FIG. 7D is an overhead perspective of the device of FIG. 7A, according to an embodiment of the invention;

FIG. 8A is a perspective of a coiled device for sampling the cervix, according to an embodiment of the invention;

FIG. 8B is a perspective of the device of FIG. 8A showing the outer perimeter of the coil with abrasive material attached, according to an embodiment of the invention;

FIG. 8C is a cross section perspective of the device of FIG. 8B contacting the epithelial tissue from the cervix, according to an embodiment of the invention;

FIG. 9A is a cross section perspective of a device for sampling the anus, showing a sharpened railing or dam around the circumference, according to an embodiment of the invention;

FIG. 9B is a cross section perspective of the device of FIG. 9A inserted into the anus showing the direction of rotation, according to an embodiment of the invention;

FIG. 10A is a cross section perspective of a coiled device with a handle attached inserted within a catheter into the cervix, according to an embodiment of the invention;

FIG. 10B is a cross section perspective of a coiled device for sampling the cervix with a handle attached inserted inside the cervix within a catheter, according to an embodiment of the invention;

FIG. 10C is a cross section perspective of a coiled device for sampling the cervix contacting the epithelial tissue from the cervix with the catheter withdrawn from the cervix, according to an embodiment of the invention;

FIG. 11 is a cross section perspective of a balloon device with a backstop for insertion into a body cavity, according to an embodiment of the invention;

FIG. 12A is a micrograph of tissue collected from the ear of the inventor using an embodiment of the invention; and

FIG. 12B is a drawing of the micrograph of tissue shown in FIG. 12A, collected using an embodiment of the invention.

### DETAILED DESCRIPTION OF THE INVENTION

### Definitions

The transitional term ‘comprising’ is synonymous with ‘including,’ ‘containing,’ or ‘characterized by,’ is inclusive or open-ended and does not exclude additional, unrecited elements or method steps.

The transitional phrase ‘consisting of’ excludes any element, step, or ingredient not specified in the claim, but does not exclude additional components or steps that are unrelated to the invention such as impurities ordinarily associated with a composition.

The transitional phrase ‘consisting essentially of’ limits the scope of a claim to the specified materials or steps and those that do not materially affect the basic and novel characteristic(s) of the claimed invention.

As used herein, the term ‘abrasive material’ refers to one or more of fabric, strands of wire, twisted strands of wire, strands of fibers, twisted strands of fibers, extruded fibers or hooks. The fabric can be composed of one or more of metal, plastic, organic polymers and inorganic polymers. The strands of wire can be composed of one or more of metal, plastic, organic polymers and inorganic polymers. The strands of fibers can be composed of one or more of metal, plastic, organic polymers and inorganic polymers. The extruded fibers can be composed of one or more of metal, plastic, organic polymers and inorganic polymers. The hooks can be composed of one or more of metal, plastic, organic polymers and inorganic polymers. In an embodiment of the invention, a composite fabric, wire, fiber, extruded fiber or hook can be formed by attaching a layer of an organic polymer onto an inorganic substrate. Abrasive material includes ‘toothbrush’ bristles (as in a bristle brush design), steel wool, corrugated plastic, Velcro® which is used to describe fabric hook and loop fastener and Kylon® which refers to abrasive material with fenestrated loops attached to a base. As used herein, the term ‘fenestrated loop’ refers to a hooked, ‘candy-cane’ shape formed by severing a loop, where a short, hooked end is less than approximately 50% of the length of the loop. In an embodiment of the invention, a fenestrated loop is formed by severing a loop once, leaving a short arm adjacent to the fenestrated loop. A Kylon fenestrated loop, in comparison to a Velcro fabric hook, is relatively longer overall. Further, the hook portion of a fenestrated loop is shallower than the Velcro fabric hook. The longer strands can more easily flex and splay on contact with tissue contact, allowing frictional abrasion in a rake like motion when rotating or sweeping the fabric across the tissue surface. The rake effect will also scoop tissue into the fabric base during tissue excavation, thus forming a collection receptacle in the fabric base. A fenestrated loop can be used for excavational frictional epithelial biopsies.

A ‘metal’ comprises one or more elements consisting of lithium, beryllium, boron, carbon, nitrogen, oxygen, sodium, magnesium, aluminum, silicon, phosphorous, sulphur, potassium, calcium, scandium, titanium, vanadium, chromium, manganese, iron, cobalt, nickel, copper, zinc, gallium, germanium, arsenic, selenium, rubidium, strontium, yttrium, zirconium, niobium, molybdenum, technetium, ruthenium, rhodium, palladium, silver, cadmium, indium, tin, antimony, tellurium, cesium, barium, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold, mercury, thallium, lead, bismuth, polonium, francium and radium. A wire means one or more of a loop of wire, a segment of wire, a metal ribbon, a metal strand or an un-insulated wire, an animal string, a fiber, a plastic, and a polymer.

A ‘plastic’ comprises one or more of polystyrene, high impact polystyrene, polypropylene, polycarbonate, polymethylmethacrylate, low density polyethylene, high density polyethylene, polypropylene, acrylonitrile butadiene styrene, polyphenyl ether alloyed with high impact polystyrene, expanded polystyrene, polyphenylene ether and polystyrene impregnated with pentane, a blend of polyphenylene ether and polystyrene impregnated with pentane or polyethylene and polypropylene, Teflon, polymer impregnated Teflon, and polysuflides.

An ‘organic polymer’ includes natural organic polymers and synthetic organic polymers. Natural organic polymers include wood, paper, cotton, wool and cellulose. A synthetic organic polymer comprises a material synthesized from one or more reagents selected from the group comprising of styrene, propylene, carbonate, ethylene, acrylonitrile, butadiene, vinyl chloride, vinyl fluoride, ethylene terephthalate, terephthalate, dimethyl terephthalate, bis-beta-terephthalate, naphthalene dicarboxylic acid, 4-hydroxybenzoic acid, 6-hyderoxynaphthalene-2-carboxylic acid, mono ethylene glycol (1,2 ethanediol), cyclohexylene-dimethanol, 1,4-butanediol, 1,3-butanediol, polyester, cyclohexane dimethanol, terephthalic acid, isophthalic acid, methylamine, ethylamine, ethanolamine, dimethylamine, hexamthylamine diamine (hexane-1,6-diamine), methyl methacrylate, pentamethylene diamine, methylethanolamine, trimethylamine, aziridine, piperidine, N-methylpiperideine, anhydrous formaldehyde, phenol, bisphenol A, cyclohexanone, trioxane, dioxolane, ethylene oxide, adipoyl chloride, adipic, adipic acid (hexanedioic acid), sebacic acid, glycolic acid, lactide, caprolactone, aminocaproic acid and or a blend of two or more materials synthesized from the polymerization of these reagents.

An ‘inorganic polymer’ includes natural inorganic compounds and synthetic inorganic polymers. Natural inorganic compounds include silicon, molybdenum silicide, silicon dioxide, silicon carbide, silicon sulfide, silicon nitride, ferrosilicon, silicon-germanium, germanium dioxide, polysilazanes, polyphosphazenes, polyphosphazene chloride, polydichlorophosphazenes. Synthetic inorganic polymers include alkoxy silanes, allyltrimethoxysilane, tetramethylorthosilicate, tetraethylorthosilicate, polydimethylsiloxane, polydimethylsilylene, polycarbosilane, polymethylhydroxysiloxane, polydiphenylsiloxane, polythiazyl, polydibutylstannane, boron nitride ceramics.

A ‘head’ is that part of a device used to sample epithelial tissue, where abrasive material is associated with the head and the abrasive material contacts the epithelial tissue. The head can be composed of one or more of metal, plastic, organic polymers and inorganic polymers. A ‘facet’ is a surface in the head of a biopsy device, where the surface's contour differs from the contour of the head of the biopsy device. The term ‘facet’ is used in analogy to a facet of a gem, where the gem facet has a surface contour that differs from the other surface contours of the other facets of the gem. A facet that is cut at an angle of thirty (30) degrees relative to the major axis of the head of the biopsy device is equivalent to a ‘point’ cut in a gem that can produce one side of an octahedron. A facet that is cut at an angle of three (3) to nine (9) degrees relative to the major axis of the head of the biopsy device can be thought of as equivalent to one of the thirty (30) odd cuts in a gem's crown to produce a ‘brilliant’. In contrast to the facet of a gem which is typically flat, the facet cut in the head of a biopsy device can have a flat, concave or convex surface contour. That is a flat facet of a biopsy device has neither a positive nor a negative radius of curvature. A convex facet of a biopsy device has a positive radius of curvature relative to the flat facet. A concave facet of a biopsy device has a negative radius of curvature relative to the flat facet. The curvature of a cylinder or rod will be referred to as positive in contrast to the negative curvature of a concave facet cut into the cylinder or rod. The curvature of a convex facet cut into the cylinder or rod will be referred to as positive.

A ‘railing’ is a thin wall that substantially or completely encircles a facet on the head of a device. The railing can be used as a dam to retain adhesive that adheres the abrasive material to the facet. An abrasive railing is a railing in which the outer edge of the railing forms the circumference of the head and has one or more sharpened, roughened, corrugated or saw tooth textures cut into the edge to allow the railing edge to abrade tissue. A device can incorporate an abrasive railing at a portion of the head that is proximal to the biopsy site the edge parallel to the fabric can achieve the scraping when the rotational motion is applied and a non-abrasive railing or rounded outer edge at the portion of the head that is distal to the biopsy site. The height of the railing can protrude from the head but protrude less than the height of the abrasive material. When the device is in contact with the tissue surface, the semi-rigid fabric hooks can splay and bend, allowing the railing to contact the tissue surface as well. Pressure directly applied to the tissue and torque pressure will shave or excavate tissue which will be swept by the fabric hooks and collected into the fabric base.

A ‘backstop’, ‘disc stopper’ or ‘hilt’ is a protrusion that in an embodiment of the invention can be associated with a head. In an embodiment of the invention, a surface of the backstop can have abrasive material associated with the backstop. In an embodiment of the invention, the distal surface of the backstop closest to the site of tissue sampling can have abrasive material adhered to the surface. In an embodiment of the invention, the backstop encircles the main axis of the head. In an embodiment of the invention, the position of the backstop relative to the head can be altered to allow the backstop to slide towards the site of tissue sampling. A ‘device shaft’ or ‘handle’ is a grip which can be attached to a head of a device used to exert pressure on the head at a distance. In an embodiment of the invention, a user can easily decouple the device head from a device shaft. Following biopsy, the head of the device can then be readily severed from the handle to allow the head to be deposited in a liquid fixative agent. In an embodiment of the invention, the handle material is scored (thus weakened) near the head to allow the head to be broken off from the handle and deposited in liquid fixative, which is usually formaldehyde or alcohol. The abrasive material, fibers, and/or device head (all with the tissue entrapped between the fibers) can be removed from the vial of liquid fixative to remove the tissue from the head of the device and process it for analysis. The liquid fixative can be formaldehyde or alcohol. The abrasive material with the tissue entrapped can be removed from the vial of liquid fixative to remove the tissue and process it for analysis. For example, in an embodiment of the invention, the shaft can be inserted into the head via a clip or screw thread mechanism, a key-in-lock design with a pressure release button, or a luer-lock type of attachment. Once the biopsy is obtained, the head and handle/shaft parts can be decoupled, where the handle can be discarded, or sterilized and re-used, and the head immersed in a vial of fixative. In various embodiments of the present invention, the tip of the devices fractures from the tip with bending pressure, allowing it to be placed into the vial of fixative for preservation and easy transport.

In the following description, various aspects of the present invention will be described. However, it will be apparent to those skilled in the art that the present invention can be practiced with only some or all aspects of the present invention. For purposes of explanation, specific numbers, materials, and configurations are set forth in order to provide a thorough understanding of the present invention. However, it will be apparent to one skilled in the art that the present invention can be practiced without the specific details. In other instances, well-known features are omitted or simplified in order not to obscure the present invention.

Parts of the description will be presented in data processing terms, such as data, selection, retrieval, generation, and so forth, consistent with the manner commonly employed by those skilled in the art to convey the substance of their work to others skilled in the art. As is well understood by those skilled in the art, these quantities (data, selection, retrieval, generation) take the form of electrical, magnetic, or optical signals capable of being stored, transferred, combined, and otherwise manipulated through electrical, optical, and/or biological components of a processor and its subsystems.

Various operations will be described as multiple discrete steps in turn, in a manner that is helpful in understanding the present invention; however, the order of description should not be construed as to imply that these operations are necessarily order dependent.

Various embodiments will be illustrated in terms of exemplary classes and/or objects in an object-oriented programming paradigm. It will be apparent to one skilled in the art that the present invention can be practiced using any number of different classes/objects, not merely those included here for illustrative purposes.

Systems and methods in accordance with embodiments of the present invention can provide for improved presentation and interaction with digital content and representations of digital content. Representation as used herein includes, but is not limited to, any visual and/or audible presentation of digital content. By way of a non-limiting example, digital images, web pages, digital documents, digital audio, and other suitable content can have corresponding representations of their underlying content. Moreover, interfaces such as graphical user interfaces can have corresponding representations of their underlying content.

The invention is illustrated by way of example and not by way of limitation in the figures of the accompanying drawings in which like references indicate similar elements. It should be noted that references to ‘an’ or ‘one’ embodiment in this disclosure are not necessarily to the same embodiment, and such references mean at least one.

Abrasive Material

In various embodiments of the invention, a head with an abrasive surface and/or associated abrasive material can be used to secure tissue samples from epithelial layers.

The frictional sampling material of the invention can be collectively referred to as abrasive material. In an embodiment of the present invention, the abrasive material can be a fabric. In an embodiment of the present invention, the fabric contains hooks. In an embodiment of the present invention, the fabric can be Kylon®. Kylon has been more fully described in U.S. Utility application Ser. No. 12/669,638 filed Jan. 19, 2010, published as 2010/0210968, the National Phase of Patent Cooperation Treaty Application US08/70341 filed Jul. 17, 2008 which claims priority to U.S. Provisional Application No. 60/950,280 filed Jul. 17, 2007 and U.S. Utility Application Ser. No. 13/072,773 filed Mar. 28, 2011, U.S. Utility application Ser. No. 13/072,775 filed Mar. 28, 2011 and U.S. Provisional Application No. 61/318,128, filed Mar. 26, 2010. These applications are herein expressly incorporated by reference in their entireties.

In a preferred embodiment, the abrasive material can be arranged to efficiently capture tissue. Thus, in one preferred embodiment, the abrasive material can be arranged in an orderly orientation. For example, the abrasive material can be arranged in rows between which the tissue can be captured. The abrasive material can be arranged to be oriented at approximately the same angle and direction. In addition, the spacing between abrasive material can be made to be the same or different.

In use, the device can be oriented so that the abrasive material can be perpendicular to tissue, and then pressure can be applied. As a result, the epithelial surface can be frictionally sheared. Thus, the abrasive material can be preferably mounted on a flat or curved platform, optimally four (4)-ten (10) millimeters in diameter so as optimize this process. However, alternatively shaped platforms can also be used in certain embodiments. Because the abrasive material can be mounted directly on the platform, which can be flat or slightly curved, the orientation remains evenly spaced and the spaces inside the abrasive material and between them remain evenly distributed to facilitate tissue capture.

In an embodiment of the invention, the abrasive fabric can be attached or sewed into another fabric or material such as the finger of a glove, with the human finger or hand functioning as the applicator to frictionally press and abrade the tissue surface.

In an embodiment of the invention, the abrasive fabric can be derivatized with functional groups to bind specific marker molecules present on cells of interest. PCT Application Number: PCT/US2009/053944, published as WO20120019920, entitled “Porous Materials for Biological Sample Collection” to Zenhausern et al, which describes an inorganic material which can be used as the abrasive material rather than for example the nylon which is used in Velcro to allow the specific binding and/or the solubilizing of the abrasive material with appropriate solvents and which is incorporated by reference in its entirety.

In an embodiment of the invention, the abrasive fabric can be cut into various shapes and interlaced into a pattern by placing the fabric on a flat, concave, convex, curved, conical or corrugated surface. The orientation or direction of the hooks can be angled in different directions on the same platform so that the hooks can abrade and collect epithelium regardless of the direction of the frictional rubbing, rotation or back and forth motion applied by the physician, practitioner or examiner performing the biopsy.

### Areas to Be Sampled

Internal body cavity surfaces in addition to the oral cavity or lower genital tract can be sampled include: oropharynx, larynx and bronchial tree, mediastinum under endoscopy, upper and lower gastrointestinal tract via endoscopy, the anus and anal verge, uroepithelium from the kidney calyx, ureter, or bladder under endoscopy, the joint surfaces or synovium during arthroscopy, internal lower genital tract including the endometrial cavity, and many organ surfaces under laparoscopy including but not limited to the ovary, uterine serosa, fallopian tubes, peritoneal cavity surface, and serosa of any intraperitoneal organ including but not limited to stomach, liver, intestine, and omentum. Retroperitoneal structure surfaces such as kidney, adrenal, and lymph, and soft tissue surrounding the structures can also be sampled. Intravascular lumen cavity can also be sampled under endoscopy. Alternatively, other tissue harvesting and collection technology such as abrasive material can excavate, collect, and store samples to be tested for DNA.

In various embodiments of the present invention, the materials used to make either the head and/or the abrasive materials are chosen such that the abrasive material can be secured to the head. In an embodiment of the present invention, the materials used to make the head and the abrasive material are chosen such that the abrasive material can be ultrasonically welded to the head. In an embodiment of the present invention, the head and the abrasive material are made of the same material such that the abrasive material can be ultrasonically welded to the head. For example, nylon abrasive material can be ultrasonically welded to a nylon facet implanted in the head. Alternatively, nylon abrasive material can be ultrasonically welded to a nylon head. In another embodiment of the present invention, the abrasive material can be extruded through injection molding during the process of injection molding the head. In an alternative embodiment of the present invention, the abrasive material can be attached to the head using an adhesive. For example, an ultra violet (UV) light activated adhesive can be used to affix the abrasive material to the head. A railing can be introduced onto the facet of the head and the UV light activated adhesive can be placed within the confines of the dam made by the railing.

In an embodiment of the invention, the railing allows sufficient adhesive to be retained in the dam so that the abrasive material can be securely adhered to the head. In an unexpected result, using a railing and adhesive to adhere the abrasive material significantly decreased the amount of abrasive material that was shed or broken off from the head during quality testing when the abrasive hooks are purposefully stressed under excess pressure intended to dislodge hooks that are partially sheared on the margin of the fabric adjacent to the device edge. Even though nylon, which can be used as the composition of the abrasive fabric, can be nontoxic, increasing the securing of the hooks enhances patient safety. In this manner, the railing and the ability to dam the adhesive so that the adhesive bound the abrasive material to the head increased the amount of tissue retained using the sampling head.

In an embodiment of the invention, the edge of the railing can be roughened or sharpened such that the rough or sharp edge acts to (a) abrade cells, (b) acts as a wall that guides manufacture and/or placement of the pad, and (c) ultimately forms a glue dam that anchors the abrasive material securely to the head. In an alternative embodiment of the invention, the edge of the railing has abrasive material attached to it and the glue dam wall anchors the abrasive material adhered on the facet to the abrasive material at the edges; this will, in and of itself, abrade tissue creating a receptacle boat into which the abrasive material resides and collects and retains the sample cells for removal from the body. In an embodiment of the invention, the railing and the edge can be retracted into the body of the head when abrasion by the edge is not required and raised out of the body of the head when abrasion by the edge is required. In an embodiment of the invention, the height of the railing can be adjusted so that the degree of abrasion is optimized for the appropriate angle with which the head contacts the epithelial surface. In an embodiment of the invention, the adjustment of the height of the railing can be effected by pneumatic pressure through the catheter. In an alternative embodiment of the invention, the adjustment of the height of the railing can be effected by gearing driven by an electric motor.

In various embodiments of the invention, depending on the angle at which the head interacts with the tissue being sampled, the rough or sharp edge can act to aggressively abrade cells while the hooks of the abrasive material can gently abrade. Accordingly, the abrasive material also serves as a receptacle for the scraped epithelial tissue.

In an embodiment of the invention, after the glue is applied to the head, the glue dam wall (a) acts to seat the cut pieces of abrasive material within the walls of the dam like a puzzle piece in a puzzle receptacle; (b) allows the excess glue to flow over the dam walls onto the edge of the abrasive material, coating the peripheral hooks at the cut edge of the abrasive material, strengthening the attachment of the abrasive material to the head and preventing dislodgement of either the abrasive material and/or the tissue cells collected during abrasion of the tissue surface; and (c) allows the head to be used to aggressively abrade the tissue surface as desired by raising and abrading the edges of the glue dam by angling the wall so that the “cutting” surface is used; by roughening the plastic; by corrugating the top of the glue dam wall or by making a saw tooth at the top surface of the glue dam wall. The saw tooth or rough edge of the glue dam wall is rotated on the surface of the tissue, dislodging epithelium via shaving or abrasion, and the hooks sweep the tissue into the abrasive material. The walls create a “boat” with the abrasive material at the base so that when the tissue is dislodged by the abrasive glue dam wall, the tissue cells are swept into the cavity of the boat and can be easily retained. In an embodiment of the invention, the tissue filled abrasive material in the boat dramatically enhances tissue collection, retention in the device and removal.

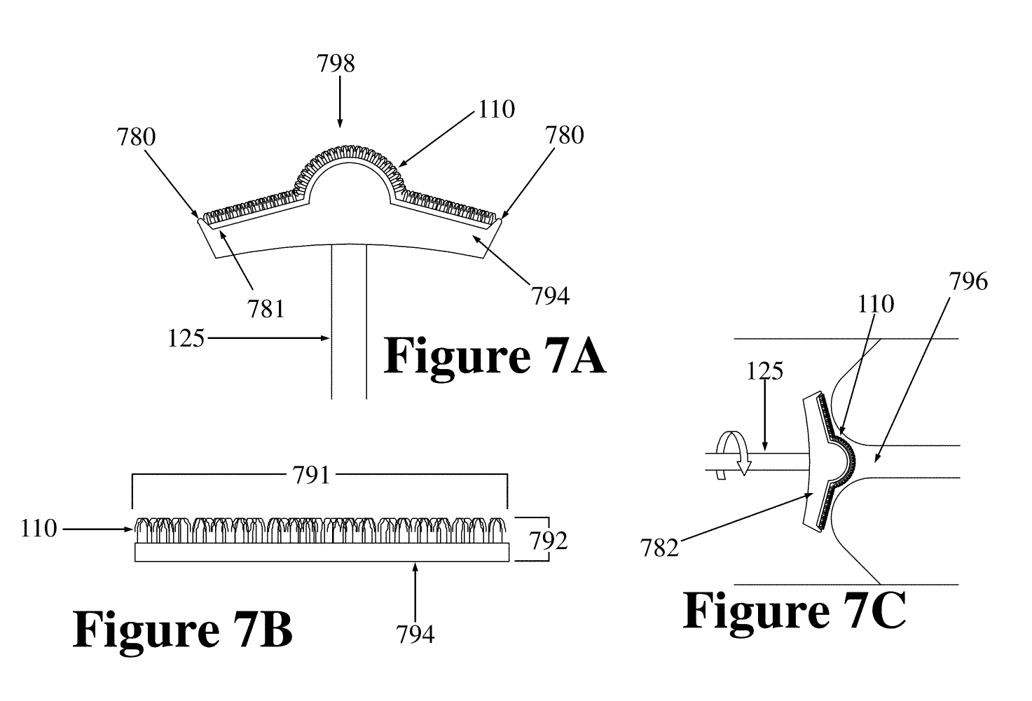

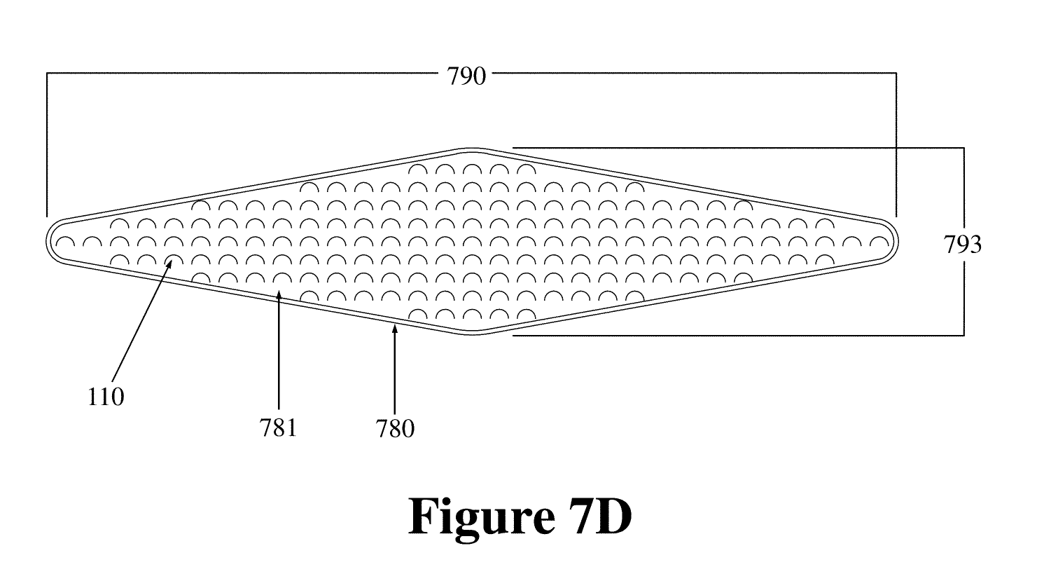

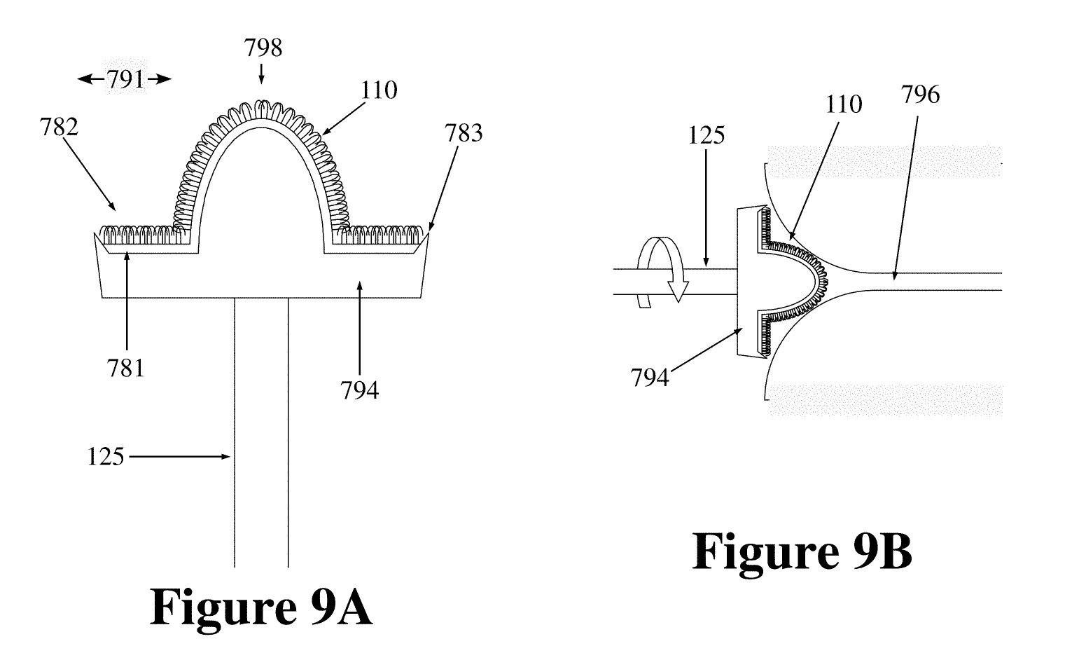

In various embodiments of the invention, the tissue obtained can be of sufficient thickness to allow a histologic sample to be retrieved. In various embodiments of the invention, the head collects cells for cytological analysis and tissue suitable for histologic analysis. In an embodiment of the invention, the abrasive material applied to the head can be used to take an Anal Cytological or deeper Histological grade biopsy (Anal Col). In an embodiment of the invention, the Anal Col can be a test used for the early detection of anal cancer. The Anal Col can provide deeper samples of tissue. The Anal Col can be a hybrid of cytology and histology excavating deeper tissue samples than is possible with conventional spatula or bristle brush technology, and providing the added advantage of collecting and retaining the tissue obtained. In various embodiments of the invention, some free floating cells and some intact partial or full trans-epithelial tissue strips or fragments can be dislodged and collected in the abrasive material base, and retained in the boat receptacle created by the glue dam walls. In an embodiment of the invention, regarding any epithelial lesion of interest for biopsy, a hub fiber or “pin” penetrates and anchors the center of the disc on the target biopsy area, serving as a central post upon which to rotate the disc. The hub or pin increases the stability and therefore allows for greater accuracy to be provided while rotating the head on the target biopsy area as there is less or no migration of the biopsy surface from a targeted lesion. In another embodiment of the invention, the Anal Col can be conical in shape with a central post for insertion into the anal cavity to just beyond the anal verge, yet remain in contact with the outer skin, optimally sampling at the squamo-columnar junction of the anus. In an embodiment of the invention, an Anal Col head includes a hub or pin that is higher and deeper, shaped like a nipple with a disc being about ¼ inch to ½ inch wide, to which the fabric strip with rounded edges can be attached. In an embodiment of the invention, the Anal Col head can have an abrasive glue dam railing. In an embodiment of the invention, the nipple of the Anal Col is so much higher and deeper than the standard version, allowing the post or hub to get deeper into the anal canal in the region of the “transformation zone” of the anus where the glandular rectal cells meet the squamous anal epithelium, also called the anal verge, i.e., the distal end of the anal canal, forming a transitional zone between the skin of the anal canal and the peri-anal skin. These anatomic landmarks also have been described for the cervix as the squamo-columnar junction where various embodiments involving a conical nipple shaped device can be used (see FIG. 7A and FIG. 9A). Alternatively, the device can be a rectangular strip with a protrusion or bump in the center that can invaginate into the orifice of a hollow canal structure, with the glue dam or railing surrounding its entire periphery. This can be similar to cutting a strip shape rectangle across the median plane of the nipple shaped device, leaving a structure as described above.

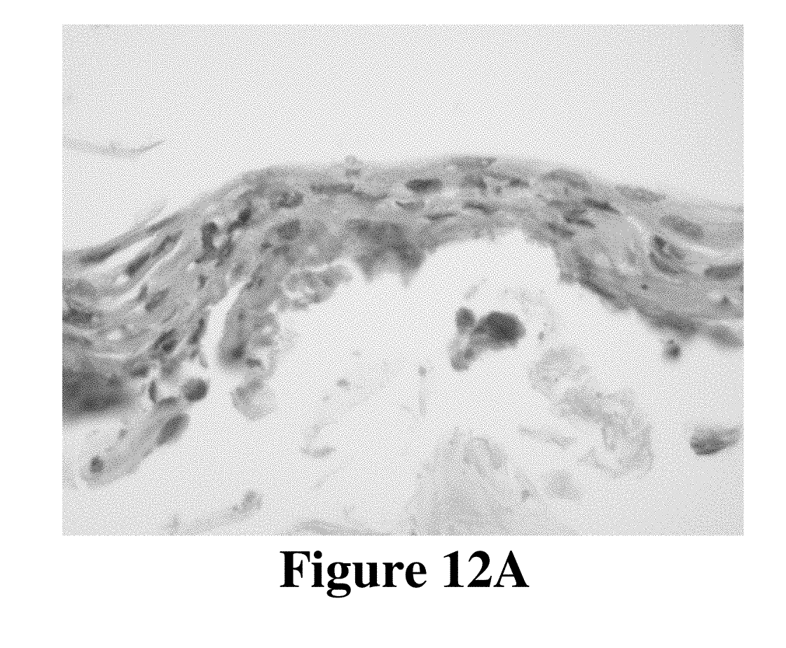

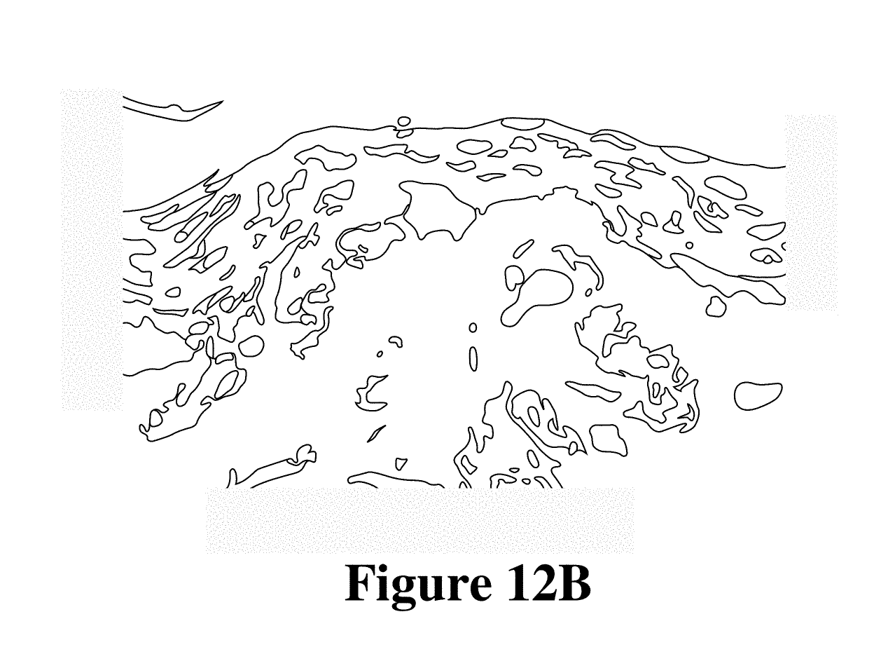

In various embodiments of the invention, the abrasive material applied to a head can be used to obtain a biopsy of epithelial tissue from the esophagus, bowel, anus, oropharynx, bladder, ureter, bronchial tree, joint space, synovial cavity, blood vessel lumen, cardiac chamber surface, mediastinal cavity structures, spinal canal, cerebral ventricles, cheek, gum, palate, sinuses, larynx, oropharyngeal structure, buccal biopsy and skin biopsy including the ear canal. In various embodiments of the invention, the abrasive material applied to a head can be used to obtain a biopsy sample of epithelial tissue in areas where underlying bone or cartilage provides resistance. In various embodiments of the invention, the abrasive material applied to a head can be used to obtain a biopsy sample of soft tissue where tensioning muscle can be used to provide resistance. In various embodiments of the invention, the abrasive material applied to a head can be used to obtain a biopsy of epithelial tissue from any body surfaces and cavity. FIG. 12A shows a micrograph of tissue collected from the ear of the inventor using Kylon applied to a 5/16″ slightly domed (convex) facet head, where the head was inserted into the ear on the bony area near the ear canal and rotated five (5) times clockwise and anticlockwise while applying moderate pressure (similar to the dentist approved pressure used to brush teeth) with an embodiment of the invention FIG. 12B is an artist's drawing of the micrograph of tissue shown in FIG. 12A.

In an embodiment of the invention, a gastrointestinal biopsy, a bladder biopsy, or any other epithelial lined body cavity that has a lumen can be biopsied either using a free standing unit placed in body cavity in advance or a unit passed through a trochar or port on an endoscope. In an embodiment of the invention, the head can be made of or can contain material that is opaque to x-ray, fluorescence, magnetic waves, sound waves or ultrasound analysis to allow the biopsy head to be x-ray or ultrasound, fluorescence, Magnetic Resonance Imaging (MRI) or x-ray guided. In an embodiment of the invention, the level of esophagus can be visualized through a catheter, after the throat is numbed, while monitoring the position of the head to the appropriate level using an appropriate monitoring technique and the opaque marker. For example, a gas (e.g., air, carbon dioxide, or nitrogen) or fluid inflating a balloon can be observed as an echo lucent (permitting the passage of ultrasonic waves without echoes) patch under ultrasound analysis. In contrast, clear fluid and air are both sonolucent allowing the passage of ultrasound waves without reflecting them back to their source. Once the abrasive material containing head is positioned at the appropriate location, then the balloon can be inflated to bring the abrasive material into contact with the biopsy the area. This allows a four quadrant or a complete circumferential ring shaped biopsy to be taken simultaneously.

In an embodiment of the invention the patches of abrasive material project radially out from the balloon. The head can be advanced in front of an endoscope as a separate unit, or can be guided endoscopically, using MRI, x-ray, or ultrasound. In an embodiment of the present invention, the tip of the device proximal to the abrasive region can contain a hollow chamber or repository containing sonolucent air or fluid. In an embodiment of the present invention, the tip of the device proximal to the abrasive region can contain an ultrasound sono denseradio-opaque marker. In an embodiment of the present invention, the ultrasound sono denseradio-opaque marker can be used so that the ancillary radiologic or ultrasound technology can be used to easily locate the active biopsy portion of the device in concert with locating the lesion or area of the biopsy. In an alternative embodiment of the invention, a RFID (radio frequency identification) tag is placed in the head to localize the head in vivo. The operator can mark the distance from the oral cavity entrance to the lesion biopsy site (depth of insertion).

In an embodiment the biopsy device is equipped with a light source, a miniature camera and remote transmission capability so that the unit itself can serve to directly visualize the lesion or anatomical area status during the biopsy procedure.

### Biopsy of a Sessile Polyp or Pedunculated Polyp of a Hollow Viscus

In an embodiment of the invention, the head can be collapsed, passed through an endoscope port and then expanded, exposing the abrasive fabric to the biopsy site. Attempting to pass the head through the port in its open state, can result in the hooks being bent, sheared off, or damaged.

Pedunculated polyps of the gastrointestinal, reproductive, urological, synovial and other body spaces or cavities are usually removed using a snare like shearing or cutting device because the stalk allows detachment by removing the lesion across a small body surface area, with minimal vascular or perforation risk, Perforation of a hollow viscus or body cavity can be catastrophic leading to bleeding or infection. Sessile polyps or lesions on hollow viscus body cavity walls such as the large colon prevent trans-epithelial biopsies because of the risk of perforation. Rarely is the entire lesion biopsied or excavated in its entirety for fear of risk of perforation or other trauma. In an embodiment of the invention the fabric can be applied to the sessile polyp (exopytuci) or ulcerated lesion (endophytic) either by direct contact with an applicator platform or a semi-rigid platform such as an inflated balloon (fabric adherent to the balloon surface) to frictionally abrade and collect the lesional tissue in a safer and more minimally invasive approach. Because the fabric will not lacerate tissue, it will attenuate the epithelial surface to the basement membrane leaving the stromal and serosal tissue intact, thus preventing perforation of the viscus.

Protect Hooks from Portal of Entry Sheath or Collapsed

In an embodiment of the invention, balloons that are deflated can be used, then the wand unsheathed, with the sheath being used to cover the biopsy hooks, then slid back or removed when the device enters the body cavity. After biopsy, the sheath can be replaced over the tip gently, so as not to dislodge tissue, and then the unit can be withdrawn through a port or trochar. In an embodiment of the invention, the device can also be lever based. A balloon with a rigid tube and opening to allow the fabric to be pushed to one side or the tip, while still being anchored to the “wand” or tube can give the operator some freedom to apply torque or pressure to the balloon.

In an alternative embodiment of the invention, a catheter can be threaded through a tube and its opening after the device is inserted. In an embodiment of the invention, the skinny sheath is pushed through the port first, followed by the catheter with its abrasive material tipped balloon, umbrella, or other small applicator.

In another embodiment of the invention, the catheter tip with the abrasive material is covered with a hinged clamshell-like covering to allow protected, insertion, and removal of the catheter tip into an endoscopic channel or sheath.

In another embodiment of the invention, a forceps-like device opens radially 180 degrees like a fan, or 360 degrees as a complete circular probe coated with biopsy fabric, and then closes down on itself, so the narrow closed fan can be pulled out through the portal inside the sheath. The unit is sufficiently rigid to apply force to the biopsy tip surface or rotate it on the lesion and collect tissue.

In various embodiments of the invention, the head can be used during laparoscopy, pelviscopy, thoracoscopy, midistinoscopy, bronchoscopy, cystoscopy, retrograde pyeloscopy, anoscopy, sinus endoscopy, gastrointestinal tract endoscopy, or arthroscopic surgery to abrade soft tissue therapeutically or diagnostically (biopsy). In various embodiments of the invention, the head can be used to screen tissue otherwise unreachable, such as an ovarian surface; this allows a histological sample to be taken for Ovarian Cancer Screening. In various embodiments of the invention, the head can be used to debride internal wounds (clean them, like a scrub brush). The debrided wound can also be biopsied. In various embodiments of the invention, the head can deliver a therapeutic agent to the wound to treat a malady. In various embodiments of the invention, the head can contain an antibiotic, antiseptic, clotting agent, enzyme substance to denature proteins, and other tissue substances.

In various embodiments of the invention, the head can be used to excavate and biopsy plaque from arterial walls or debride and remove endovascular clots via thrombectomy during vascular catheterization surgeries or interventional radiologic procedures.

In various embodiments of the invention, the head can contain one or more abrasive surfaces including Kylon, free fabric like sandpaper, and sandpaper. In various embodiments of the invention, the abrasive fabric can be applied to a glove, to an applicator, to foam plastic, to rubber, to a sanding unit, or to a padded sanding unit.

In another embodiment applied to sampling non-mucosal keratinized epithelial surfaces such as skin and peri-anal skin, the keratin layer can create a barrier to complete trans-epithelial biopsy. Kylon has been shown to take biopsies to at least the level of the stratum corneum layer when pressed with moderate pressure and rotated at least three (3) complete 360 degree revolutions in a clockwise and counterclockwise buffing manner, Winter M. et al., J. Low Genit. Tract Dis., vol. 16: pp. 80-87 (2012) Fabric-Based Exocervical and Endocervical Biopsy in Comparison With Punch Biopsy and Sharp Curettage which is hereby explicitly incorporated by reference in its entirety.

In various embodiments of the invention, the head can biopsy skin; can be used to monitor therapy, genomics, proteomics, metabolomics, aging and microbiologic status; and can be used to measure toxins or drug concentrations at the tissue level, monitor markers associated with tissue damage and aging, and other features of epithelium.

There are many possible uses for different embodiments of the present invention. An embodiment of the invention can be adapted for local or regional biopsies, or simultaneous biopsies of epithelial surfaces and canal-like structures. An embodiment of the invention can be used as a frictional tissue sampling and collection biopsy device and as a method of immune response by autoinoculation. An embodiment of the invention can be used for drug application and cervical biopsies equipped with a radio frequency identification tag for recording the details and other conditions of use. Details are discussed below.

### Focal Biopsy

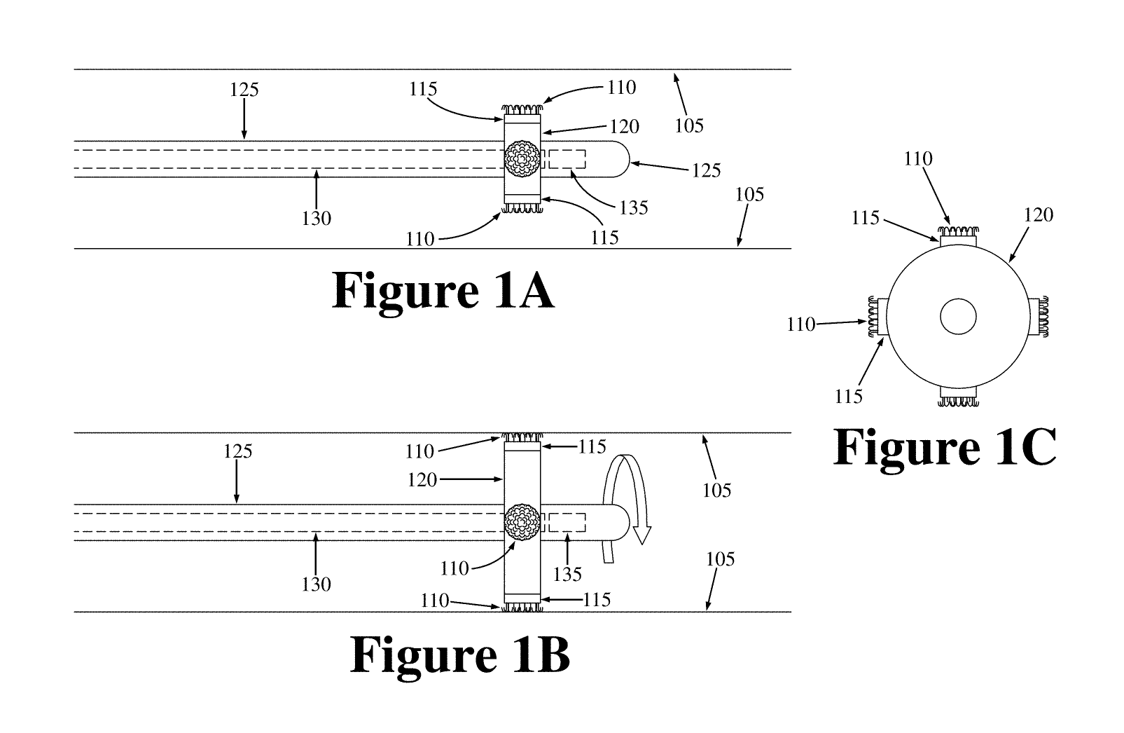

In an embodiment of the invention, Some embodiments relate to a trans-epithelial, frictional tissue sampling and collection device can be used to perform biopsies of lesions suspected of harboring disease. In an embodiment of the invention, a lesional biopsy site is no larger than about ten (10) millimeters in diameter (i.e., focal biopsy). In an embodiment of the invention, the lesions are accessible to the physician, practitioner or examiner during routine examination. In another embodiment of the invention, the surface is accessible following entry into a body cavity through a natural orifice or surgical channel via a trochar and inspection using an endoscope with a biopsy port. The device head is inflated or otherwise expanded or placed to rest on the lesion or area of intended biopsy/therapy. In various embodiments of the invention, a focal biopsy apparatus is configured with abrasive material attached to a balloon. Referring to FIGS. 1-11, a focal biopsy apparatus is configured with abrasive material that is about one (1) millimeter to about four (4) millimeters in length. In an embodiment of the invention, the abrasive material is about two (2) millimeters in length.

In an embodiment of the invention, the physician, practitioner or examiner can encounter polypoid lesions on stalks of varied length. The frictional tissue sampling and collection device can capture and entangle with such polyp stalks and create a tearing or shear force which will dislodge the polyp, releasing it from its epithelial attachment and entrapping the entire polyp and stalk within the hooks and spaces between the hooks into the fabric base.

### Regional Biopsy

In an embodiment of the invention, the intent can be to biopsy and screen large geographic areas of tissue at risk for disease (e.g., neoplastic transformation such as, but not limited to, the squamo-columnar junction of the female cervix in the presence or absence of visualized lesions). The device provides samples of clumps or clusters of excavated epithelial tissue fragments for analysis. This is in contrast to other methods disclosed in prior art that provide surface and exfoliated cells by sweeping the cells from such target tissue sites, commonly with blunt spatula or soft bristle brush devices. The intent is to remove tissue based evidence with frictional biopsy of the larger area, which can range from approximately ten (10) to forty (40) millimeters in diameter. The abrasive material is purposefully pressed in a tangential manner to the tissue, in contrast to stiff bristle nylon brushes which can intentionally puncture the epithelium thus shearing or cutting such tissue. The abrasive material provides a scraping raking force with a scythe like shape. In an embodiment of the invention, the device by providing one or more concave surfaces on an otherwise conical or rod-like protrusion, allows the device to be placed on a specific location and rotated without moving off the desired location. In an embodiment of the invention, the ability to remain on a fixed location can provide samples of epithelial tissue from specific locations for analysis. In this manner, the overall surface can be randomly sampled with a finite number of biopsy samples. In contrast, other methods disclosed in prior art do not allow the position from which the biopsy is to be sampled to be localized. The intent is to frictionally remove tissue from a variety of localized positions based on visual evidence of the larger area, or knowledge of the “at-risk” landmark area where disease is likely to evolve or be harbored, such as the “transformation zone” of the cervix, which can range from approximately ten (10) to forty (40) millimeters in diameter.

Referring to FIG. 1A in an embodiment of the invention, a central disc of abrasive material 110 can be attached to a head 115 applied to a balloon 120 and inserted into a canal 105 with a catheter 125. When the catheter 125 has been inserted into the canal 105 to a depth or distance that locates the head 115 at the site where the biopsy is required, the balloon 120 can be expanded so that the abrasive material 110 rests upon one or more biopsy sites. In an embodiment of the invention, the balloon 120 can be expanded by insufflating a gas into the balloon 120 where the gas inflates the balloon 120 through an inner cannula 130 causing the head 115 and the abrasive material 110 to contact the epithelial tissue of the canal 105 (see FIG. 1B). FIG. 1B also shows that once the balloon 120 is expanded, the balloon 120 can be rotated in order for the abrasive material 110 to scrape against the epithelial tissue of the canal 105. FIG. 1C shows the cross section of the head prior to being inflated in an embodiment of the invention. A signal-opaque material 135 can be positioned in the catheter to help localize the balloon 120 in an embodiment of the invention.

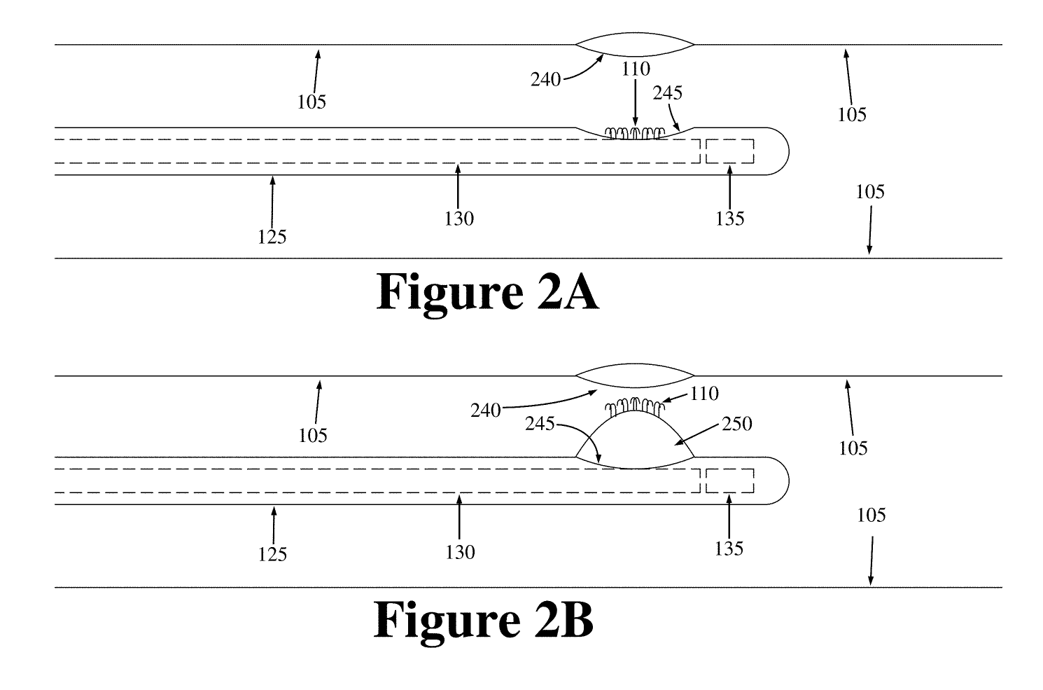

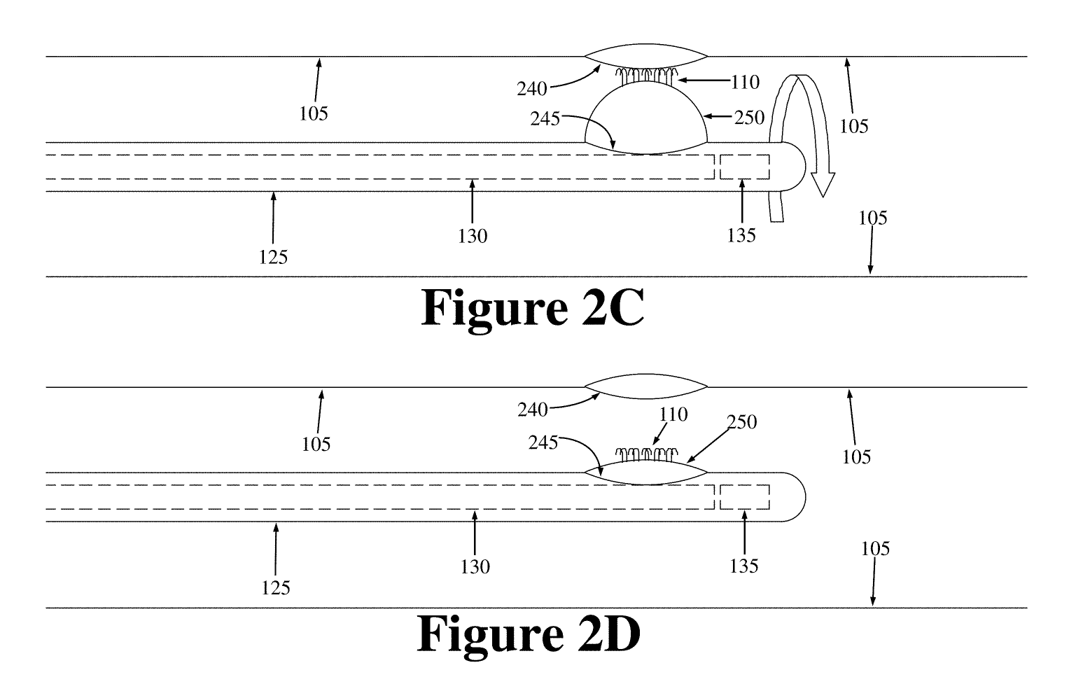

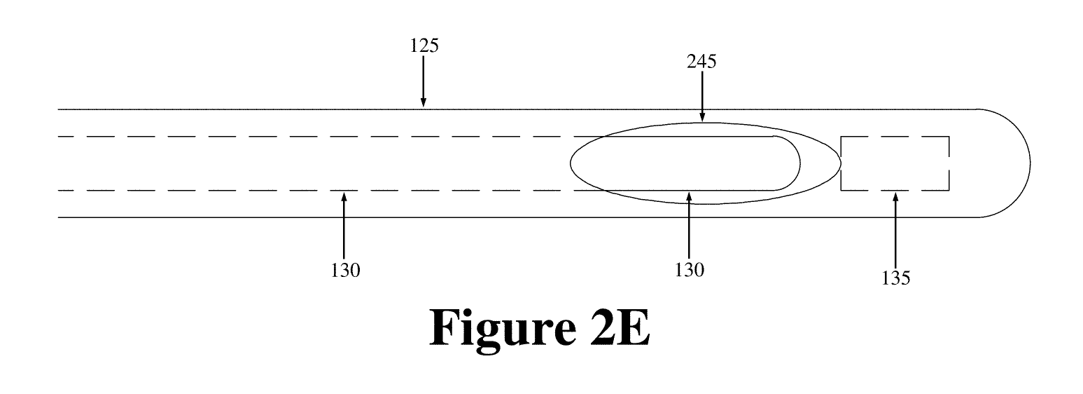

In an alternative embodiment of the invention, a catheter 125 with an opening 245 can be inserted into a canal 105 with abrasive material 110 applied to expandable material 250 see FIG. 2A. When the catheter 125 has been inserted into the canal 105 to a depth or distance that locates the opening 245 at the site where the biopsy is required 240, the expandable material 250 can be expanded so that the abrasive material 110 can be pushed out of the opening 245 by leaking a gas into the balloon 250 through an inner cannula 130 (see FIG. 2B). The expandable material 250 can be inflated so that it rests upon one or more biopsy sites 240 (see FIG. 2C). FIG. 2C also shows that once the expandable material 250 is expanded, the expandable material 250 can be rotated in order for the abrasive material to scrape against the epithelial tissue. FIG. 2D shows the expandable material n 250 can be contracted after obtaining the biopsy and the epithelial tissue withdrawn inside the opening 245. Thereafter the expandable material and the catheter can be removed to obtain the epithelial tissue. FIG. 2E shows an overhead cross section of the catheter 125 with the internal cannula 130 with the opening 245 for releasing the expandable material (not shown). A signal-opaque material 135 can be positioned in the catheter to help localize the expandable material on the biopsy site 240.

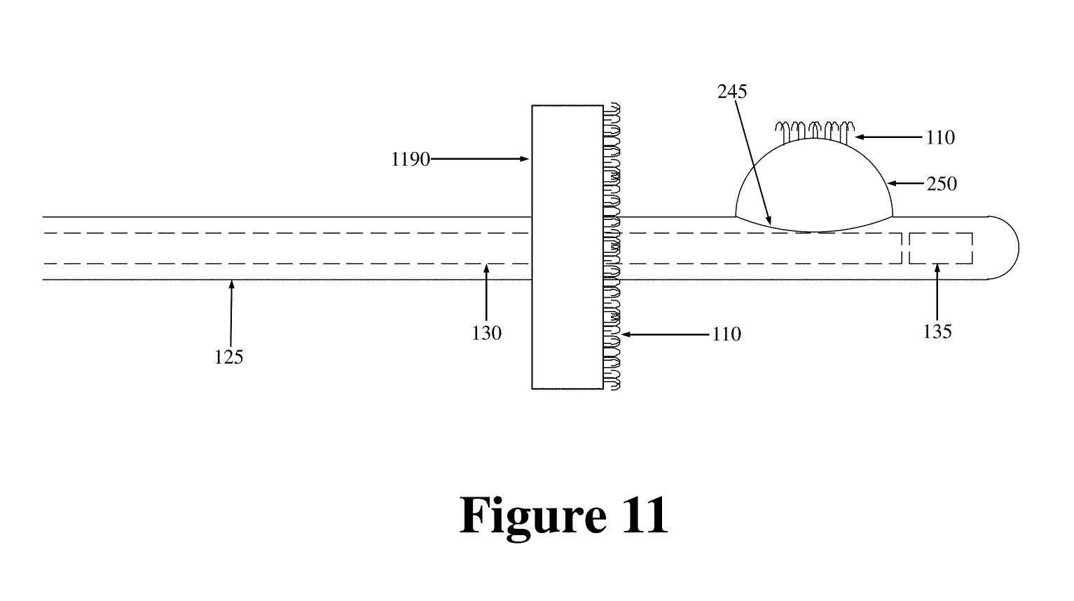

In another embodiment of the invention, a catheter 125 with an internal cannula 130, an opening 245, a signal-opaque material 135 and a backstop 1190 can be inserted into a canal (not shown) with abrasive material 110 applied to the backstop 1190 and the expandable material 250 (see FIG. 11). In addition to the abrasive material 110 on the expandable material 250, the abrasive material 110 can be applied or associated with the distal side of the backstop 1190 in order to collect a biopsy sample of more than one area simultaneously.

### Simultaneous Biopsy of Epithelial Surfaces and Canal-Like Structures

In an embodiment of the invention, the device contains a central core of abrasive material, surrounded by an expandable balloon with abrasive material. The central core is geometrically suited to insinuate within a central canal structure, such as the endocervical canal of the cervix. There is simultaneously uniform contact of the abrasive material attached to the balloon circumferentially around the endocervical canal on the flat exocervical surface. With rotation and agitation in a back-and-forth motion, tissue is harvested within the abrasive material as described above.

In an embodiment of the invention, the surface of the head has abrasive material applied. In alternative embodiments, the device has a head with longer abrasive material, surrounded by a wider rim of shorter abrasive material. The longer abrasive material is geometrically suited to insinuate within a central canal structure, such as the endocervical canal of the cervix. There is simultaneously uniform contact of the abrasive material circumferentially around the endocervical canal on the flat exocervical surface.

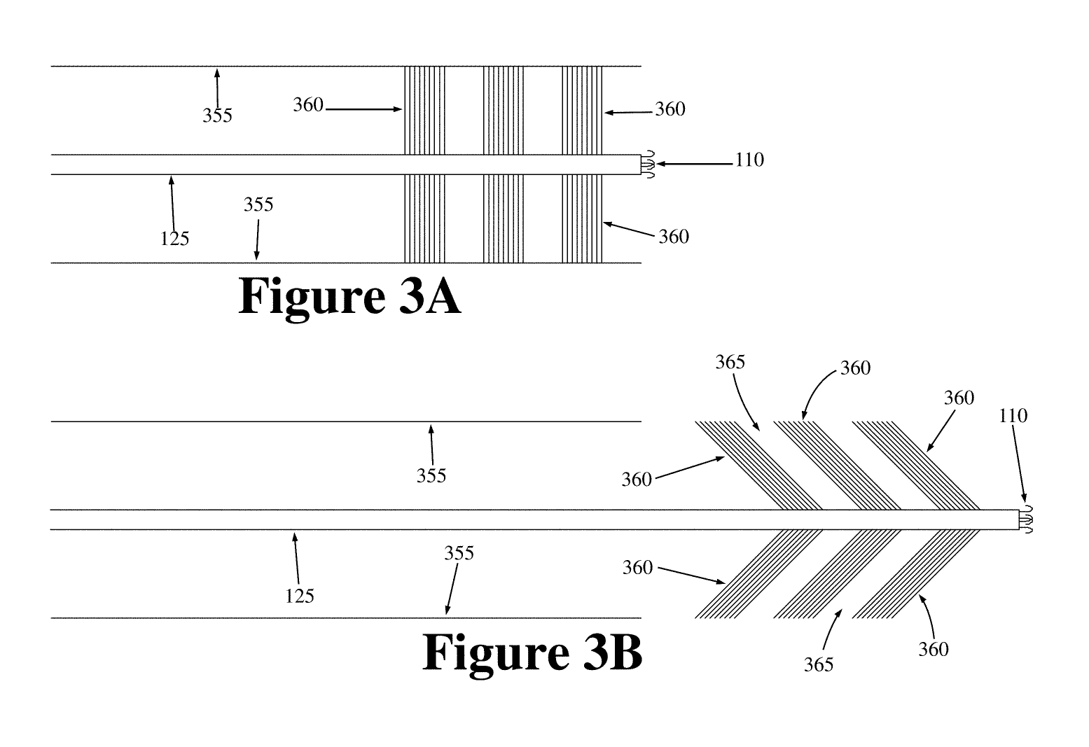

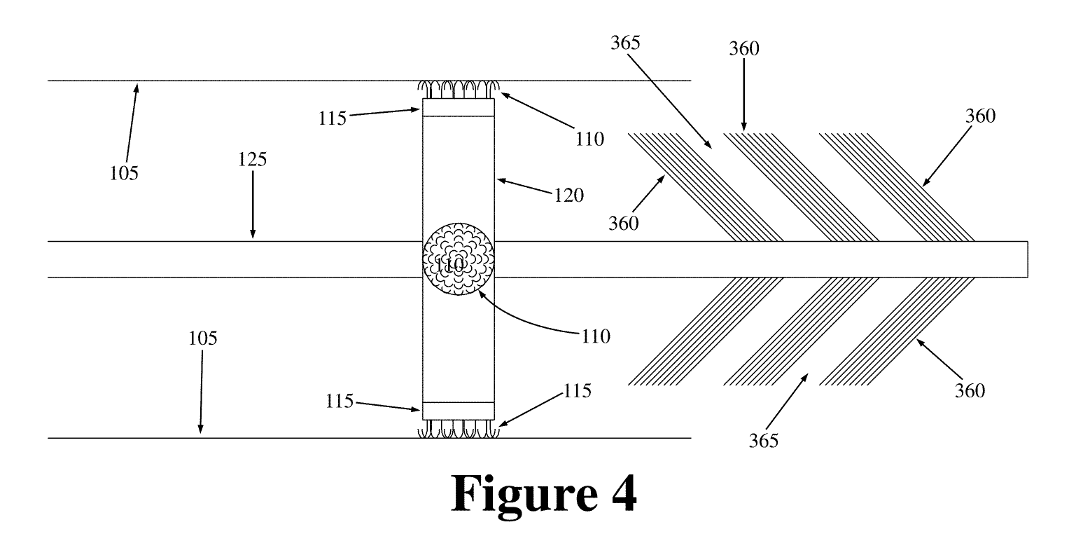

In an embodiment of the invention, a catheter 125 with an abrasive material 360 inserted inside the catheter housing 355 is fed into a canal (not shown) as shown in FIG. 3A. Once the catheter 125 is brought into position, the abrasive material 360 can be pushed out of the catheter housing 355 and contact the tissue (not shown) as shown in FIG. 3B. The catheter 125 upon which the abrasive material 360 is attached can be rotated to collect the biopsy sample. In an embodiment of the invention, a central disc of abrasive material 110 can be attached at the end of the catheter to contact tissue in a narrower canal or at the end of the canal. In an alternative embodiment of the invention, the central disc of abrasive material 110 can be attached to a small balloon which can be inflated to contact tissue in a narrower canal or the canal end (see FIG. 4). In one embodiment, a central disc of fibers which can sample at a distance of nine (9) millimeters from the center of the canal are surrounded by a central disc of fibers which can sample at a distance of three (3) millimeters from the center of the canal. An apparatus with these parameters can be inserted on/into cervix and rotated with spinning revolutions. Following frictional trans-epithelial tissue disruption, the head containing the biopsy sample can be deflated or otherwise withdrawn into the catheter housing and withdrawn.

In another embodiment shown in FIG. 3 and FIG. 4, a central disc of abrasive material 110 can be attached to a head 115 associated with a balloon 120 and inserted into a canal 105 with a catheter 125. When the catheter 125 has been inserted into the canal 105 to a depth or distance that locates the balloon 120 at the site where the biopsy is required, the balloon 120 can be expanded so that the abrasive material 110 rests upon one or more biopsy sites. In an embodiment of the invention shown in FIG. 4, the catheter 125 also has abrasive material 360 attached to contact tissue in a narrower canal or the canal end. The abrasive material 360 can be arranged into clumps with gaps 365 separating the clumps in order to collect and retain the epithelial cells dislodged by the abrasive material (see FIG. 3 and FIG. 4).

In an embodiment of the invention a catheter with a ‘backstop’ or ‘disc stopper’ covered in abrasive material is inserted into a cavity, with the catheter employing the biopsy balloon tip along its length. Once inserted, the balloon is inflated and the device rotated. This device can be used to take a biopsy of the Transformation zone of the cervix, anus, or other canal like structure where the entry portion of the canal and the inner aspects will be simultaneously biopsied. The catheter is inserted with balloon deflated recessed inside the catheter under a slit or opening in the catheter. The abrasive material is aligned under the slit opening and mounted on a balloon so that when inflated, the fabric is extruded above the plane of the catheter opening. The backstop avoids too deep an insertion into the canal, while simultaneously abutting the entry portion of the canal at the body surface. Once inserted and applied on abutment under pressure, the balloon is inflated and the fabric placed in contact under pressure to the canal wall, while simultaneously, the disc is in pressure with the canal portal of entry. When rotated 3-5 complete (360 degree) revolutions clockwise then counterclockwise, the canal and entry port area are simultaneously biopsied. The balloon can then be collapsed into the catheter cavity along with the tissue sample, and the disc removed from the application site. The tip of the device with canal and portal of entry biopsies can be cut from the catheter into a vial of fixative for later removal and pathological analysis. By ensheathing the device, the biopsy coil can be more easily inserted and removed and the patient would not sense pain or discomfort from the procedure due to a smooth covering sliding inside and withdrawn instead of a rough coil.

### Frictional Tissue Sampling and Collection Biopsy Devices

The abrasive material can be pressed in a rotational manner (e.g., in sweeping or circular motion) away from or toward the operator, perpendicular, or at an angle into epithelial tissue surfaces. In an embodiment of the invention, the abrasive material is semi-rigid and the force flexes the abrasive material. The force applying the material to the tissue creates frictional forces sufficient to cause local heating and buckling of the epithelial surface away from the underlying stroma. On pressing and rotation across the tissue surface, the abrasive material scrapes, buckles and shears the epithelium from the underlying stroma. The fragments are excoriated from the tissue surface through the concomitant application of frictional forces applied to the tissue surfaces by the abrasive material. The frictional forces overcome the adhesive and binding forces of the tissue below to release fragments of various shapes and size, all eligible for collection in a histology lab, and subsequent processing and analysis. In an embodiment of the invention, the abrasive material is Kylon and the semi-rigid fibers remain flexible enough to cause separation of the fenestrated ends, creating frictional forces sufficient to cause local heating and buckling of the epithelial surface away from the underlying stroma.

The abrasive material holds the tissue fragments after excoriation. In addition, spaces between the abrasive materials retain excoriated tissue. The frictional forces exceed the binding forces afforded by adhesion molecules which anchor epithelia to the basement membrane, as well as disrupting Van der Waals forces

Once the epithelium is frictionally sheared from the underlying stroma, the tissue clumps and epithelial fragments are swept, excavated and entrapped within the geometrically suited spaces between the abrasive material. Once the epithelium is frictionally sheared from the underlying stroma, the tissue clumps and epithelial fragments are swept and excavated by the distal most curved apex of the abrasive material and entrapped within the geometrically suited spaces between the closed abrasive material. Thus, the method is frictional abrasion, excavation via rotation and other directional motion, and tissue collection.

The abrasive material or fabric can be cut into uniform shapes such as a hybrid diamond-pear shape, a pear shape or a circular disc or straight edge shape(s) and with uniform height, allowing the device to provide 360 degree coverage of tissue surfaces over suspected lesions, without a gap within the circumference of the device. This is in distinction to bristle brushes which are spiral or bent in shape, which present surface gaps that do not allow uniform contact with the target tissue, and gaps that cause migration of the device from the lesion site toward the direction of rotation when such devices are pressed onto lesions and rotated or moved for tissue harvesting. The abrasive base material is also flexible to allow the material to be applied to a balloon that can adopt a concave or convex shape.

In an embodiment of the invention, removal of tissue from the abrasive material can include rinsing under pressure, immersion and agitation manually or mechanically, or by sonication. Alternatively, the abrasive material can be sheared from the head on telfa or other filter paper, and the abrasive material plucked off the paper leaving the entire biopsy specimen. Alternatively, after tissue is collected into the device channels, tissue can deposit via rotation or agitation in a vial of liquid fixative, rinsed off the device under pressurized spraying, or removed from the nylon fibers by cutting away the nylon fibers from the fabric (e.g., onto filter paper), thus leaving the tissue on the paper, which can be immersed in fixative.

In one aspect, the present invention relates to a frictional trans-epithelial tissue apparatus. In an embodiment of the invention, the abrasive material comprises three (3) millimeter material adhered to and projecting perpendicularly from a platform. The abrasive material can be constructed from plastic, metal, or another stiff material.

The abrasive material is of sufficient flexibility to withstand frictional forces and not fracture, and of sufficient tensile strength to generate sufficient frictional shear force during a sweeping or circular motion of the device to remove epithelium from tissue. Spaces between the abrasive material can serve to capture and harbor the sampled tissue.

In an embodiment of the invention, designed for taking a biopsy of focal lesions, a flat, flexible platform, which anchors the abrasive material can be of any size, but is most practically approximately five (5) to ten (10) millimeters in diameter and circular in shape. In an alternative embodiment of the invention, the shape of the abrasive material can be oval or another geometrical design that affords an advantage in covering the target tissue area for sampling. The platform can be hinged in such a way that it can be folded or compressed, inserted through a small endoscopic channel, and then reinstated to its original state as a platform with a sampling surface. It can be comprised of plastic, cloth, or another composite material. The abrasive material projects away from the platform towards the tissue surface. In various embodiment of the invention, a hub fiber or “pin” penetrates and anchors the center of the disc on the target biopsy area, serving as a central post upon which to rotate the disc. The hub or pin increases the stability and therefore allows for greater pressure to be applied while rotating the head on the target biopsy area.

In other embodiments intended to screen a larger, regional tissue site at risk for neoplastic transformation or other disease process, the optimal shape is circular, the diameter can range from about ten (10) to fifty (50) millimeters, and the abrasive material can project at varied distances from the platform towards the tissue surface. For the purpose of histological screening to detect cervical neoplasia, the central five (5) millimeters diameter disc projects longer (five (5) to twenty five (25) millimeters) abrasive material fibers, and is surrounded circumferentially by the aforementioned approximately three (3) to twenty three (23) millimeters long abrasive material fibers. The longer fibers insinuate inside canal structures, (e.g., the endocervical canal) simultaneously with contact of the shorter fibers with an outer endothelial surface (e.g., the exocervical surface). Upon pressure and rotation or agitation, the endocervical and exocervical tissues can be simultaneously frictionally sheared and collected. Histological screening can be necessary to correctly reflect the presence or absence of epithelial pathology, because adhesion molecules can prevent representative exfoliation from diseased tissue in some cases, leaving cytological screening methods lacking in accuracy.

Preferably, a frictional trans-epithelial biopsy sample is taken from an anatomical region that is to be tested.

In an embodiment of the invention, a plastic, metal, or mixed composition cannula can be threaded through a catheter.

In an embodiment of the invention, a head made of any suitable material (e.g., wood, plastic, paper or metal) from which an expandable balloon can be inflated, which has the abrasive material adhered to the head or otherwise associated or incorporated. The cannula and head are constructed large or small depending on the size of the catheter. The cannula can be rigid or semi-rigid so as to not bow or arc when pressure is transmitted to rotate the head.

A handle into which the cannula can be transfixed is optionally mechanical, providing motorized rotational, drill-like movement or agitating vibration. The device handle can be composed of stiff material, including plastic similar to Lucite, clear or opaque in coloration, rigid nylon plastic, or alternatively can be glass, wood or metal. The device head can take a variety of shapes, cylindrical or tapered in design, but the distal most surface face is circular, square, or polygonal, and can be composed of plastic (e.g., nylon). The device head diameter can range from approximately five (5)-fifty (50) millimeters. The abrasive material fabric can be welded to the nylon surface ultrasonically, or can alternatively be attached via adhesive, or via a rim or collar (e.g., which snaps on to the surface into a recess in the head of the device).

In an embodiment of the invention, the operator examines tissue surfaces and chooses an area to be sampled based on the investigation being performed. In other embodiments, the operator chooses an anatomical landmark known to be “at risk” for neoplastic or disease transformation for the purposes of sampling the entire chosen surface.

With moderate pressure, the physician, practitioner or examiner simultaneously inflates and thereby applies pressure to the epithelial tissue and rotates the device against the tissue several times in a clockwise or counterclockwise direction, opening or separating the abrasive material, thus performing frictional disruption of the tissue surface. With moderate pressure, the physician, practitioner or examiner simultaneously presses and rotates the device against the tissue several times in a clockwise or counterclockwise direction, or agitating motion in alternating 75-120 degree rotations, clockwise and counter clockwise. These actions cause an opening or separating of the abrasive material, thus performing frictional disruption of the tissue surface. Alternatively, a sweeping motion can be used. If a motorized handle is used, it can be activated to assist in the rotation or vibration of the device.

The harvested tissue is collected from the tissue surface, and some tissue already trapped in abrasive material can be inspected and teased from the abrasive material, or the abrasive material separated from the balloon, and placed in a fixative solution.

The frictional tissue sampling and collection device can be used on any body surface, both external to the body, body cavities, or on internal organs. To access epithelial surfaces of internal body organs, the device head can be deflated, folded or collapsed to pass through a small aperture or port, and reopened or expanded to fully expose the fabric to the biopsy surface. This device can be used on humans or any other living organism with an epithelial surface. Any tissue surface can be sampled. The ease of use in each case will be related to the strength of the individual tissue adhesion and binding forces in specific locations. The abrasive material can harvest the tissue and also serve as tissue collection reservoirs for later storage once placed in a fixative medium. The abrasive material can be detached from any applicator for later examination and processing (i.e., decoupled from the instrument used to press against tissue surfaces to obtain the tissue sample).

If the tissue surface is a canal or concave shaped area of the body, instead of a perpendicular platform design, the abrasive material can be directly attached to the probe itself which can be gradually tapered at the end to facilitate insertion into the canal. The abrasive material can project perpendicularly from the probe surface at its distal end, and the unit, once placed into the canal that is lined on its surface with epithelium, contacts such epithelium snugly.

The abrasive material can be mounted on the platform or project from the rim surface of the platform, perpendicular or at an angle to the platform along the margin of the platform, or attached to other delivery applicators, including the physician, practitioner or examiner's gloved finger, or other surgical instruments. The platform can be any shape or size which can fit on a tissue surface. The base assembly can be any shape or size, and can be permanently rigid or collapsible.

If the tissue surface lies within a canal shaped tissue surface, the abrasive material can be attached directly to the applicator probe, which can be inserted into the canal shaped body cavity. The probe with the abrasive material projecting from the surface and contacting the epithelium is rotated causing the frictional disruption sampling from the tissue surface. The shape of the probe can be constructed in any shape that allows a snug fit into the canal. The abrasive material can be arranged in rows or equally spaced, allowing for maximal contact and tissue collection.

In an embodiment of the invention, motorized mechanical assistance can be used to take the biopsy via a mechanical handle into which the most proximal end of the cannula can be inserted. Such mechanical assistance can enhance the rotational or vibratory force that the device transmits to the tissue after contact is established. This can increase the frictional forces and the speed of the tissue disruption/sampling and shorten the procedure time.

Some methods for removal of tissue from the fiber assembly include using a brush, rinsing under pressure, immersion and agitation manually or mechanically, or by sonication. Alternatively, the fibers can be sheared from the fabric on telfa or other filter paper, and the fibers plucked off the paper leaving the entire biopsy specimen. Alternatively, after tissue is collected into the device channels, tissue can be deposited via rotation or agitation in a vial of liquid fixative, rinsed off the device under pressurized spraying, or removed from the nylon fibers by cutting away the nylon fibers from the fabric (e.g., onto filter paper), thus leaving the tissue on the paper, which can be immersed in fixative.

In an embodiment of the invention, the abrasive material can be pressed perpendicular or at an angle to the epithelial tissue surfaces. Upon application of rotation or agitation pressure, tissue epithelial fragments can be frictionally separated from the underlying tissue basement membrane and stroma. The channels between the abrasive material entrap and collect the tissue fragments. The process is in contrast to sharp curettage where the purposefully sharp edge of the curette first incises, pierces, then shaves and scoops epithelium and underlying stroma from the tissue surface. The process described herein is less perceptible to patients than conventional biopsies and causes a smaller amount of blood loss and trauma.

The abrasive material can be of sufficient flexibility to withstand frictional forces and not fracture, and of sufficient tensile strength to generate sufficient frictional shear force during a sweeping or circular motion of the device to remove epithelium from tissue. Spaces between the abrasive material can serve to capture and harbor the sampled tissue.

Upon pressure and rotation or agitation, the endocervical and exocervical tissues can be simultaneously frictionally sheared and collected. Histological screening can be necessary to correctly reflect the presence or absence of epithelial pathology, because adhesion molecules can prevent representative exfoliation from diseased tissue in some cases, leaving cytological screening methods lacking in accuracy (see for example Lonky et al., J. Low Genit. Tract Dis. (2004) 8:285 “False-negative hybrid capture II results related to altered adhesion molecule distribution in women with atypical squamous cells pap smear results and tissue-based human papillomavirus-positive high-grade cervical intraepithelial neoplasia” and Felix et al., Am J Obstet. Gynecol. (2002) 186:1308, “Aberrant expression of E-cadherin in cervical intraepithelial neoplasia correlates with a false-negative Papanicolaou smear” which articles are herein expressly incorporated by reference in their entireties).

In an embodiment of the invention, a frictional trans-epithelial biopsy sample is taken from a lesion or an anatomical region that is predisposed to disease.

The handle or applicator probe is grasped at its proximal end or handle. The distal portion or head of the device contains the base, surface and abrasive material that project perpendicular from the base towards the tissue surface with the more rounded ends that are pressed against the tissue surface.

### Method of Inducing an Immune Response by Autoinoculation

In an embodiment of the invention, the trans-epithelial, frictional tissue sampling and collection devices described herein are utilized to agitate and disrupt epithelial cells containing a pathogen, or cellular proteins altered by a pathogen, to induce an immune response against the pathogen. This results in auto-inoculation of tissues that harbor pathogens and macromolecules such as virally altered DNA and/or oncogenic proteins. The method can also be termed therapeutic frictional abrasion-excoriation. This method is advantageous when a pathogen is normally able to evade an immune response. For example, some viruses remain in surface epithelial layers where they are sequestered from the immune system. Other viruses can be integrated into cellular DNA, thereby evading immune detection.

The methods of inducing an immune response against a pathogen that normally evades the immune system comprise the steps of a) disrupting epithelial cells containing the pathogen, virally altered DNA, or cellular oncoproteins with a micro-curettage device described herein, and b) introducing the pathogen into the bloodstream of a patient to elicit an immune response.

In an embodiment of the invention, the trans-epithelial, frictional tissue sampling and collection devices described herein are utilized to disrupt epithelial cells to induce an immune response against human papilloma viruses (HPVs). HPVs are persistent viruses that can remain in their hosts for long periods of time before causing any ill effects. Generally, the host reacts to viral pathogens by generating both humoral and cell-mediated responses. Humoral responses are typically antibody-mediated and involve the secretion of antibodies such as immunoglobulin A (IgA) and immunoglobulin G (IgG) by B lymphocytes. Cell-mediated responses, on the other hand, are carried out by immune effector cells such as dendritic cells (DCs), natural killer (NK) cells, macrophages and T lymphocytes which secrete a number of cytokines including interferons (INF) and tumor necrosis factor (TNF), and up-regulate the expression of Fas ligand (FasL) and TNF-related apoptosis inducing ligand (TRAIL) on their cell surface.

In the case of HPV infection, the immune response is frequently weak or undetectable, and accompanied by little or no inflammation. Even when an immune response is elicited, it may not be able to clear the virus. Disruption of the epithelial surface by frictional tissue disruption induces repair and inflammation, and serves to auto-inoculate the patient. Without wishing to be bound by any theory, exposure of the epithelial surface to frictional tissue disruption, uniquely induced by the apparatus and methods disclosed herein through local heating from friction forces exerted, can enhance the induction of repair and inflammation, and encourage an immune response following patient autoinoculation. Agitation or scrubbing of a lesion serves to introduce viral particles into the bloodstream of a patient where they can trigger a humoral or antibody related immune response. In addition the method can fracture cells releasing antigens locally within the tissue stroma inducing a cell mediated response associated with the release of cytokines and attraction of helper and killer T cells to the sampled tissue area.

Advantageously, the method of the present invention auto-inoculates a patient with viral particles of the specific viral serotype(s) that the patient is infected with. In contrast, current vaccine strategies are effective on a subset of HPV strains. For example, GARDASIL™ by Merck & Co., Inc. is indicated to help prevent cervical cancer, precancerous and low-grade cervical lesions, vulvar and vaginal pre-cancers and genital warts caused by HPV types 6, 11, 16 and 18 and Cervarix™ by GlaxoSmithKline is an HPV 16/18 cervical cancer candidate vaccine. The vaccine is commonly injected in a limb, not the target organ at risk, the cervix, and has been only documented to elicit a humoral antibody immune reaction.

### Dispensing Adjuvant, Drug or Active Ingredient

In an embodiment of the invention, an adjuvant drug or an immune modulating agent can be used in combination with the autoinoculation method, thus augmenting an immune response. For example, Imiquimod (Aldara® topical cream, manufactured and marketed by Graceway Pharmaceutical Company) is approved for the treatment of actinic keratosis, external genital warts and superficial basal cell carcinoma, a type of skin cancer. An immune response can be enhanced by using such immune modulating agents in combination with autoinoculation by the methods described herein. In an embodiment of the invention, the adjuvant drug can be applied to the abrasive material fibers directly akin to toothpaste on a toothbrush. In an embodiment of the invention, the adjuvant drug can be adsorbed onto the surface of the abrasive material and desorbed into the tissue during tissue disruption and sampling. In an embodiment of the invention, the adjuvant drug can be embedded into the abrasive material and incorporated into the tissue during tissue disruption and sampling. In an embodiment of the invention a channel within the applicator can be used to transmit the drug from the top of the handle by means of a squeeze bulb or syringe, through a small lumen in the center of the fabric disc, concomitant with the tissue disruption, delivering drug into the fracture crevices created during the frictional buckling and shearing process created by the device.

In an embodiment of the invention, a method of drug delivery to a pathological lesion or areas of tissue concomitantly disrupts tissue planes, creating crevices or pathways for drugs to enter via intra-epithelial and sub-epithelial spaces. This is in contrast to topical therapies, which are slowly absorbed into and through the epithelia; Intra-lesional application is more focused and requires a smaller drug dose, which in turn presents less risk of side effects. The drug, pharmaceutical or medicinal compound to be delivered can include an ablative, antibiotic, antiseptic and/or an immune modulating compound.

In an embodiment of the invention, an active ingredient is delivered via an applicator comprising an abrasive material as described herein. The active ingredient is applied in a manner akin to applying toothpaste to a toothbrush. In an embodiment of the invention, the active ingredient can be adsorbed onto the surface of the abrasive material and desorbed into the tissue during tissue disruption and sampling. In an embodiment of the invention, the active ingredient can be embedded into the abrasive material and incorporated into the tissue during tissue disruption and sampling. In an embodiment of the invention, the active ingredient can be injected through a hollow cannula. The active ingredient application apparatus can optionally have an element through which the drug is delivered (e.g., a syringe with a locking mechanism). The active ingredient is applied to a “wound” created by frictionally agitating the tissue. In an embodiment of the invention, the abrasive material can be impregnated with an active ingredient during manufacture, where the active ingredient leeches out into the disrupted tissue when the abrasive material contacts and macerates/disrupts the tissue.

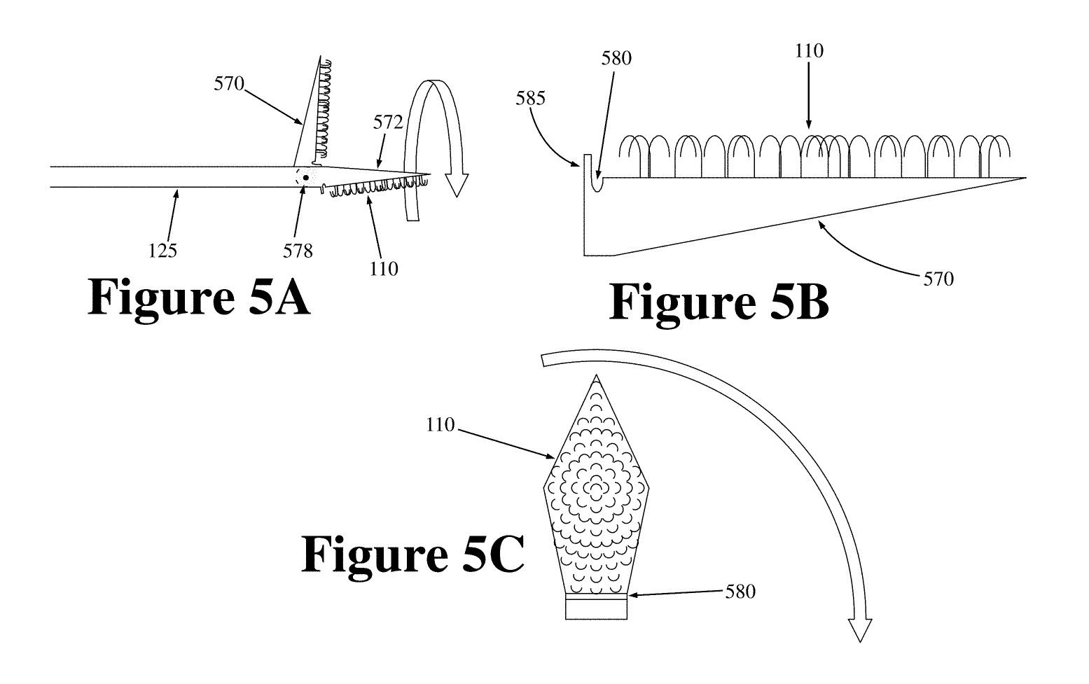

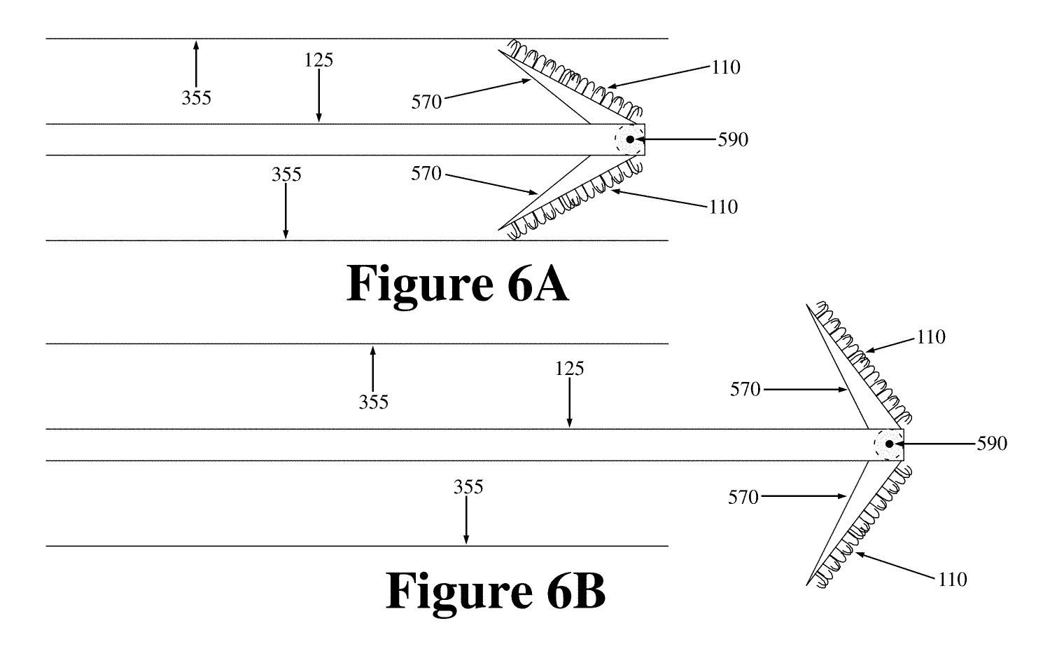

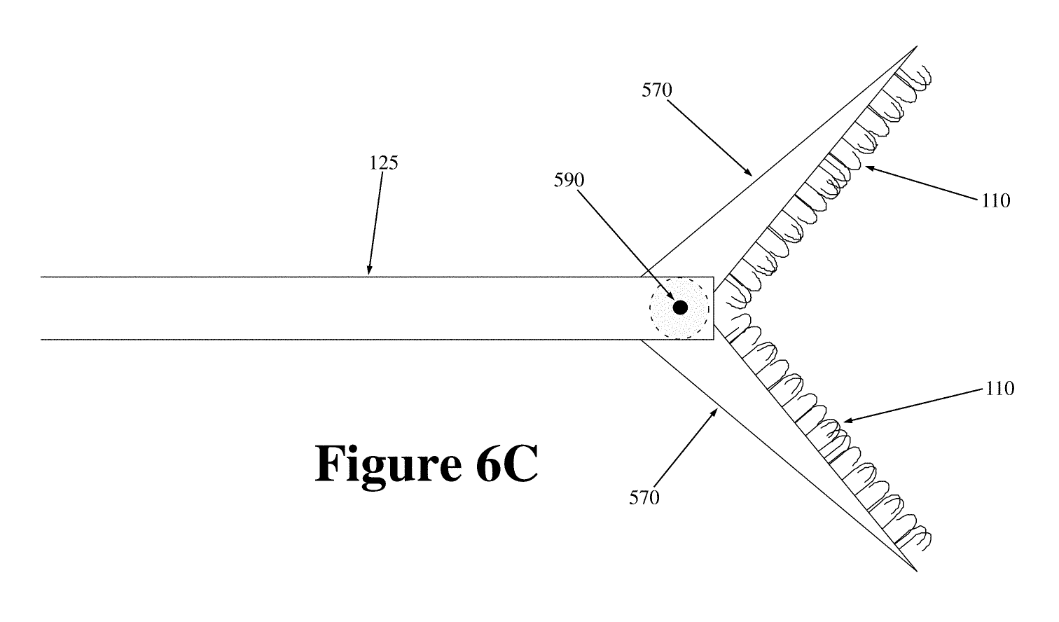

Currently a physician, practitioner or examiner can choose an exocervical or an endocervical biopsy tool. In an embodiment of the invention, a physician, practitioner or examiner can choose a hybrid exocervical/endocervical screening biopsy tool. As shown in FIGS. 5 and 6 in an embodiment of the invention, the physician, practitioner or examiner introduces a cannula 125 with the hybrid exocervical/endocervical screening biopsy tool 572 into the cervical ostium canal (i.e., the external orifice of the uterus) (not shown). As shown in FIGS. 5 and 6 in an embodiment of the invention, one or both arms 570 present on the cannula 125 can pivot around a point 590 and thereby be raised at an acute angle, perpendicular or an obtuse angle to the cannula 125 to contact one or both the squamo-columnar junction and the endocervical columnar epithelium. In an embodiment of the invention, the two arms 570 can form a convex arrangement (FIG. 6B). In an embodiment of the invention, the two arms 570 can form a concave arrangement (FIG. 6C). In an embodiment of the invention, the two arms 570 are introduced into the cervix or other canal and have a diameter of approximately the diameter of the cannula 125. In an embodiment of the invention, one of the two arms 570 is pivoted to an acute angle and the head can be rotated to collect the biopsy sample (see FIG. 5A). FIG. 5B shows a cross section of one arm 570, and the abrasive material 110 applied to the arm. A well 580 is used to allow adhesive to form a dam to adhere the abrasive material 110 to the cannula 125. The arm 570 pivots on a base 585. FIG. 5C shows the profile of the arm 570 raised perpendicular to the head (not shown), the abrasive material 110 applied to the arm, located by the well 580 and a direction of clockwise rotation (frontal perspective).

In an embodiment of the invention, a cannula 125 with the hybrid screening biopsy tool 590 can be introduced in a catheter housing 355 into a canal (not shown). As shown in FIG. 6A in an embodiment of the invention, one or both arms 570 present on the cannula 125 can pivot around a point 590 and thereby be raised at an obtuse angle to the cannula 125 in order to fit within the catheter housing 355. Once the catheter has been located at the desired site, the cannula 125 and the biopsy tool 572 can be ejected out of the catheter and the arms 570 pivoted to present an appropriate angle for the abrasive material 110 to sample the tissue (not shown). In an embodiment of the invention, the arms 570 can be pivoted around pivot point 590 to an acute angle with respect to the cannula 125 main axis and the cannula 125 can be rotated to abrade the epithelial tissue (not shown) with the abrasive material 110 to collect the biopsy sample (see FIG. 6C).

In an embodiment of the invention, a biopsy device can include a raised profile designed to simultaneously sample both the exocervical and the endocervical tissue of the cervix (see FIGS. 7A-7D). FIG. 7B shows the arm 782 with the abrasive material 110 adhered to the base of the arm 794, where the length and thickness of the arm are dimensions 791 and 792. The raised profile 798 can be partially or fully inserted into the cervix 796 to sample the endocervical tissue (see FIG. 7C), while the arm 782 can sample the exocervical tissue. In an embodiment of the invention, the arm 782 is rounded at the peripheral edges or is in the shape of a propeller blade (see FIG. 7D). In an embodiment of the invention, the arm 782 is rounded at the peripheral edges. FIG. 7D shows a frontal view of the device with the abrasive material 110 as it enters the cervical canal, where the length and width of the device are dimensions 790 and 793. In FIG. 7D, a railing 780 raises the height of the device at the perimeter leaving a lower height in the interior 781 which can be referred to as a boat. In an embodiment of the invention shown in FIG. 9A and FIG. 9B, the dimensions of the raised profile 798 relative to the length 791 of the arm 782 can be modified (c.f. FIG. 7A). In an embodiment of the invention, the perimeter of the arm 794 can have a rounded railing 783. In an alternative embodiment of the invention, the perimeter of the arm 794 can have a sharpened, roughened, corrugated or saw tooth textured railing 783 or otherwise a sharpened, roughened, corrugated or saw tooth edge cut into the perimeter of the arm 794 to allow the railing 783 to abrade tissue (see FIG. 9A). In various embodiments of the invention, the leading edge of an abrasive railing 783 can be rounded so as not to cause discomfort when sampling. The sharpened railing 783 raises the height of the device at the perimeter leaving a lower height in the interior 781 allowing the boat to collect adhesive to firmly adhere the abrasive material to the interior 781. In an embodiment of the invention, an abrasive railing 783 can achieve the scraping when the rotational motion is applied. In an embodiment of the invention, the abrasive railing 783 can be arranged at the portion of the railing 783 that is proximal to the biopsy site and a non-abrasive railing (see 780 in FIG. 7A) or rounded outer edge at the portion of the railing that is distal to the biopsy site. The height of the railing can protrude from the base 794 but protrude less than the height of the abrasive material 110. In an embodiment of the invention, when the device is in contact with the tissue surface, the abrasive material 110 can splay and bend, allowing the railing 783 to contact the tissue surface as well. Pressure directly applied via the handle 125 to the tissue and torque pressure will shave or excavate tissue which can be swept by the abrasive material 110 and collected in the boat 781.

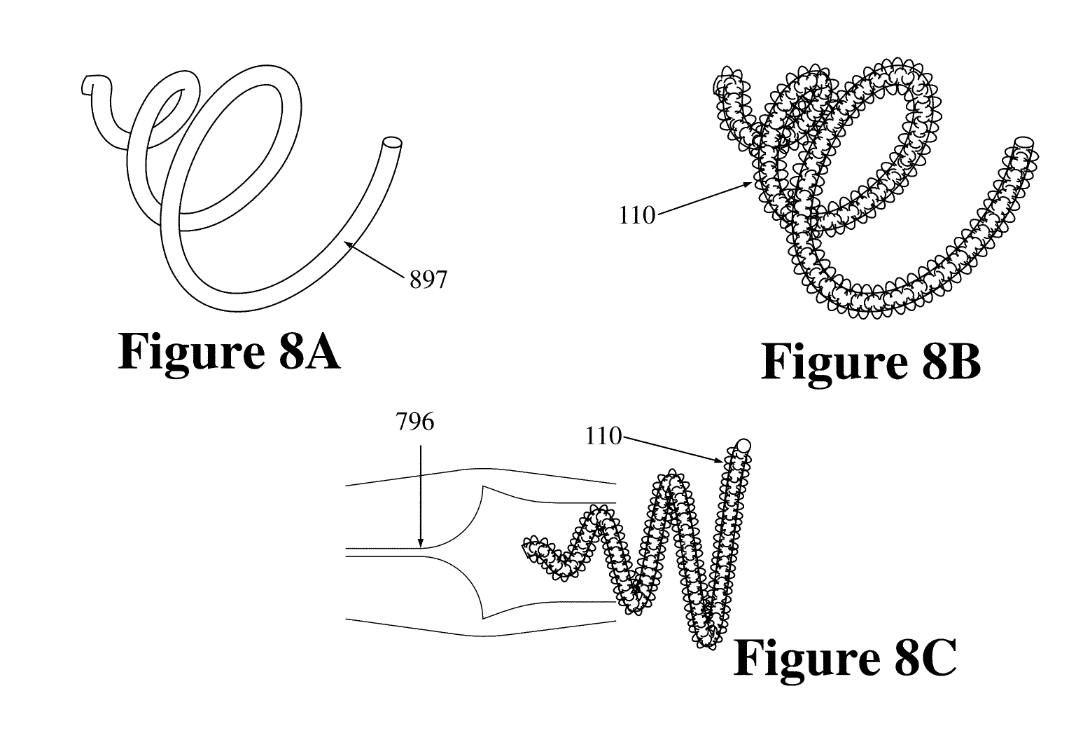

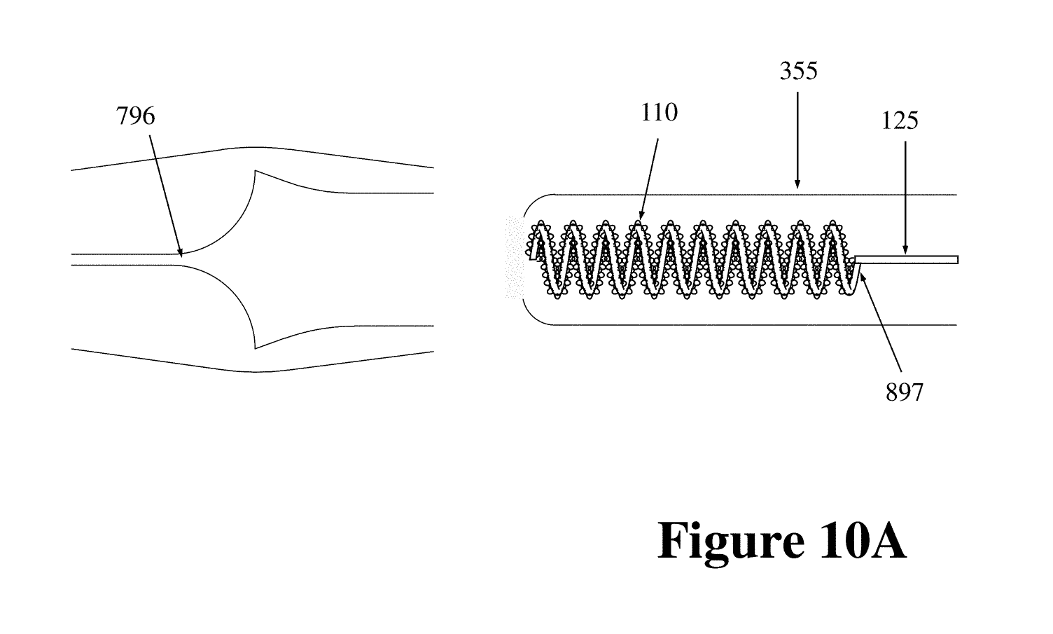

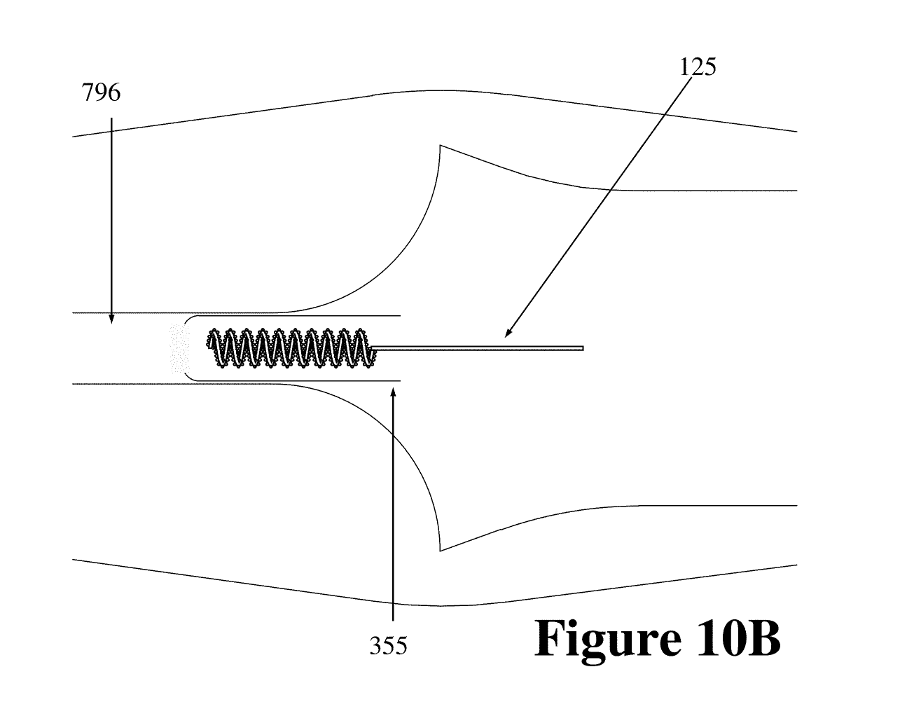

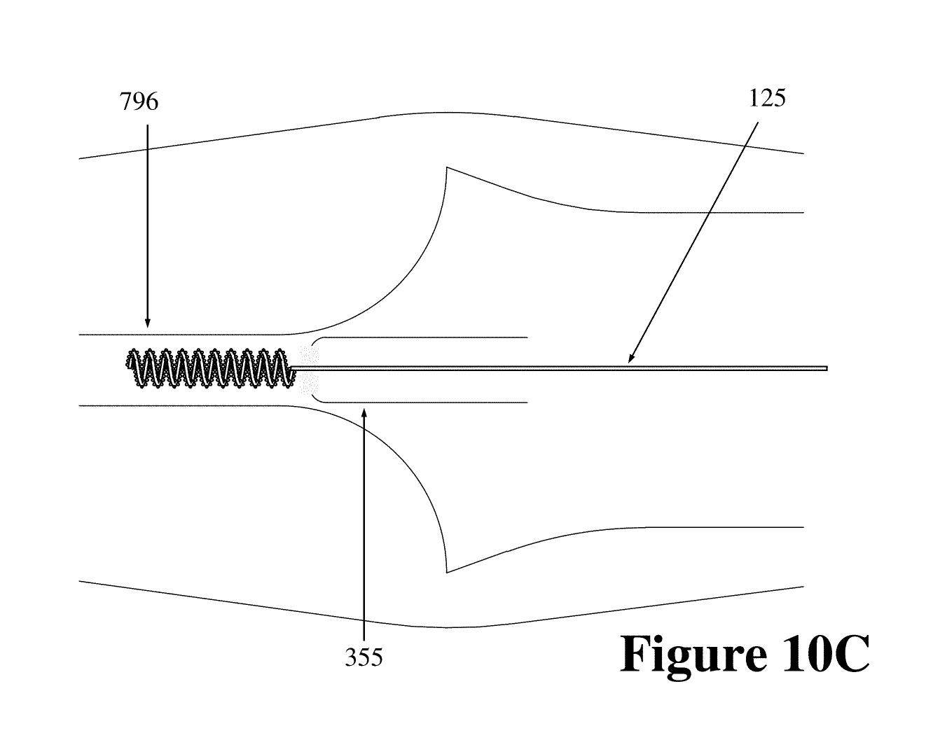

In an embodiment of the invention, a biopsy device 897 can include a coiled design with abrasive material 110 on the outer circumference of the coil to simultaneously sample both the exocervical and the endocervical tissue of the cervix 796 (see FIGS. 8A-8C). In an embodiment of the invention, the coiled biopsy device 897 with abrasive material 110 on the outer circumference of the coil can be inserted using a handle 125 into a catheter housing 355 and the catheter inserted into the cervix 796 (see FIGS. 10A-10C). The coil can be between one (1) centimeters and ten (10) centimeters depending on the length of the canal organ structure targeted by the biopsy. The backbone of the coil 897 can be made of one or more of metal, plastic, organic polymer or inorganic polymer material. The abrasive material 110 length can be from three (3) millimeters to ten (10) millimeters depending on the size of the canal like structure and the rigidity or elasticity of the walls. In embodiment of the invention, the abrasive material 110 can have hooks and the hook length can be from three (3) millimeters to ten (10) millimeters depending on the size of the canal like structure and the rigidity or elasticity of the walls (more rigid canals, more pliable hooks or fibers), more elastic walls like colon, more rigid hooks can be used to abrade the epithelial tissue. The coil 897 can have between one (1) full rotation per one (1) centimeters to one (1) full rotation per ten (1) centimeters. The diameter of the coil 897 can be between 0.5 millimeters and three (3) millimeters.

In an embodiment of the invention, a sheath or plastic shell 355 can be encased around a twisted tapered bristle brush where the brush recesses inside the sheath 355. In an embodiment of the invention, a sheath or plastic shell 355 can be encased around a head that has wings with abrasive or cutting edges, where the wings scrape the tissue when rotated, and then the abrasive material center retains the tissue. In this embodiment, the abrasive material also acts as a collecting medium and is relatively stiff. In another embodiment of the invention, a brillo brush or cotton fluff (soft Q tip) can be used in a sheath 355 for collecting the tissue inside the sheath 355. In an embodiment of the invention, the hollow receptacle contains some adhesive inside the sheath 355 or chamber for the tissue to stick or adhere to assist in retaining the cells collected. In an alternative embodiment of the invention, grooves within the sheath 355 can act like shutters that can entrap the tissue.

In an embodiment of the invention, a lesional biopsy site sampled with the device can be no larger than approximately three (3) millimeters in diameter. In an alternative embodiment of the invention, a lesional biopsy site sampled by the device can be no larger than approximately six (6) millimeters in diameter. In another embodiment of the invention, a lesional biopsy site sampled by the device can be no larger than approximately ten (10) millimeters in diameter. In an embodiment of the invention, a lesional biopsy site sampled by the device can be no larger than the diameter of the device head at a position four (4) millimeters distal from the tip. In an alternative embodiment of the invention, a lesional biopsy site sampled by the device can be no larger than the diameter of the device head at a position nine (9) millimeters distal from the tip. In an embodiment of the invention, a lesional biopsy site sampled by the device can be no larger than a focal biopsy.

Depending on the accessibility of the lesions, the choice of embodiment of the invention can be adapted to the particular situation. For example, one embodiment of the invention can be used where the lesions are accessible to the physician, practitioner or examiner in a routine examination. Another embodiment can be used where lesions are not accessible to the physician, practitioner or examiner during routine examination. In an embodiment of the invention, lesions are accessible to the physician, practitioner or examiner during routine examination. In an alternative embodiment of the invention, lesions are not accessible to the physician, practitioner or examiner during routine examination. In another embodiment of the invention, access to lesions requires surgery. In an embodiment of the invention, the tissue surface to be sampled is accessible following entry into a body cavity through a natural orifice, canal, or surgical channel. In an embodiment of the invention, the tissue surface to be sampled is accessible following entry into a body cavity via a trochar using an endoscope with a biopsy port for inspection. In another embodiment of the invention, the tissue surface to be sampled is accessible following entry into a body cavity via a cannula. In another alternative embodiment of the invention, the tissue surface to be sampled is accessible following entry into a body cavity via an arthroscope, colonoscope, sigmoidoscope, sinus scope and anoscope.

In an embodiment of the present invention, the device head remains on the lesion due to the design of the device surface. In an embodiment of the present invention, the device head remains on the immediate area of intended biopsy/therapy due to the design of the device surface. In an embodiment of the present invention, the head has a facet with abrasive material adhered to the facet. In an embodiment of the present invention, the head facet has a flat surface. In an alternative embodiment of the present invention, the head facet has a concave surface. In another alternative embodiment of the present invention, the head has a facet with a convex surface. The concave facet head allows a handle attached to the head to be rotated and ensures that the head remains on the desired location for convex tissue surfaces. The convex facet head allows a handle attached to the head to be rotated and ensures that the head remains on the desired location for concave tissue surfaces. The flat facet head with an adhered abrasive fabric allows the hand to be rotated and pressed completely without allowing gaps between the abrasion material and the surface tissue to be sampled when collecting a biopsy. In an embodiment of the invention, the head of the device is conical and pointed. In an embodiment of the invention, the head of the device is elliptical and pointed. In an embodiment of the invention, the head of the device is multifaceted and pointed.

An unexpected result that was observed during clinical trials of a number of devices, undertaken to test various prototype geometries, was that a pointed-tip rod enabled the physician, practitioner or examiner to more easily dilate the cervix, while not increasing the risk of damage to the cervix through an incision. In an embodiment of the invention, the diameter of the head of the device is a maximum of approximately eight (8) millimeters and tapers to a tip of less than approximately one (1) millimeters. In an embodiment of the invention, the diameter of the head of the device is a maximum of approximately five (5) millimeters and tapers to a tip of less than approximately one (1) millimeters. In an embodiment of the invention, the diameter of the head of the device is a maximum of approximately four (4) millimeters and tapers to a tip of less than approximately 0.8 millimeters. In an embodiment of the invention, the diameter of the head of the device is a maximum of approximately three (3) millimeters and tapers to a tip of less than approximately 0.6 millimeters. In an embodiment of the invention, the diameter of the head of the device is a maximum of less than approximately three (3) millimeters and tapers to a tip of less than approximately 0.6 millimeters.

An unexpected result observed during clinical trials was that an device with a maximum diameter of less than approximately eight (8) millimeters which tapered to a tip of less than approximately one (1) millimeters enabled the physician, practitioner or examiner to insert the device into almost any cervical canal, and then gently press to insert the device further into the cervical ostium. In many cases, the insertion also dilated the cervix to allow entry of the device deeper into the canal. This is because the device head is a smooth tapered tip which acts like a dilator. That is because the distal approximately ten (10) millimeters (corresponding to approximately one-half the length of the facet) of the device head is a smooth tapered tip it acts like a dilator. In another embodiment of the invention, because the distal approximately thirteen (13) millimeters (corresponding to approximately two-thirds the length of the facet) of the device head is a smooth tapered tip it acts like a dilator. It was unexpected that a device can be used to both dilate the cervical ostium and enter the cervix. It was also unexpected that the flatter or thinner pointed device did not significantly increase the risk of damage to the cervix by causing an incision or inadvertent puncture of collateral tissue.

In various embodiments of the invention, the pointed thin head of the device has one or more facet surfaces cut into the pointed tip to increase the area sampled in a longitudinal direction along the rod main axis. In an embodiment of the invention, the major axis of the facet surface is parallel with the major axis of the rod. In an embodiment of the invention, the minor axis of the facet surface is parallel with the major axis of the rod. In an embodiment of the invention, the one or more facet surfaces are at the distal end of the rod. In an embodiment of the invention, the widest portion of one or more of the one or more facet surfaces is at the distal end of the rod. In an alternative embodiment of the invention, the thinnest portion of one or more of the one or more facet surfaces is at the distal end of the rod. In an embodiment of the invention, one or more of the one or more facets have a concave surface. In an embodiment of the invention, one or more of the one or more facets have a convex surface.

In an embodiment of the invention, one or more of the one or more facet surfaces are diamond shaped. In an embodiment of the invention, one or more of the one or more facet surfaces are pear shaped. In an embodiment of the invention, one or more of the one or more facet surfaces are triangle shaped. In an embodiment of the invention, one or more of the one or more facet surfaces are hybrid triangle-pear-shape. In an embodiment of the invention, one or more of the one or more facet surfaces are hybrid diamond-pear-shape. The hybrid diamond-pear shaped facet surface with the diamond end distal to the handle enhances the pointed feature of the head, while the pear shaped end proximal to the handle increases surface area. Due to the tapered fit of the device into the canal orifice, the canal itself steadies the device as it is rotated, where pressure can be applied maximally to the fabric surface during rotation.

In an embodiment of the invention, the distal surface of the thin head has abrasive material attached. In an alternative embodiment of the invention, abrasive material is associated with the surface of the pointed thin head. In another embodiment of the invention, one facet surface of the pointed thin head has abrasive material adhered to the surface. In an embodiment of the invention, one or more of the one or more facet surfaces of the pointed thin head has abrasive material applied. In another alternative embodiment of the invention, two or more facet surfaces of the pointed thin head have abrasive material applied.

In an embodiment of the invention, the length of the facet on the device tip is approximately nineteen (19) millimeters long. In an embodiment of the invention, one or more of the one or more facet surfaces begins at the tip of the device head and extends towards the handle. In an embodiment of the invention, the diameter of the head four (4) millimeters distal from the facet tip is approximately two (2) millimeters. In an embodiment of the invention, the diameter of the head nine (9) millimeters distal from the facet tip is approximately 2.5 millimeters. In an embodiment of the invention, the diameter of the head twelve (12) millimeters distal from the facet tip is three (3) millimeters.

In an embodiment of the invention, the maximum overall diameter of a device with one facet is the sum of the maximum diameter of the head and the length of the abrasive material attached to the facet. In an embodiment of the invention, the overall diameter of a device at a point with one facet is the sum of the diameter of the head at that point and the length of the abrasive material attached to the facet.

In a device with maximum diameter three (3) millimeters and with abrasive material comprising fibers that are approximately three (3) millimeters in length, if the distal four (4) millimeters of the head is inserted then the device tip including the abrasive material has a diameter at this point four (4) millimeters distal from the tip) of approximately five (5) millimeters. In an embodiment of the invention, the diameter of the head greatly facilitates access into the cervical ostium. In this embodiment, the cervix needs be dilated less than approximately five (5) millimeters in order for the distal four (4) millimeters of the facet of the device to enter the cervical cavity. It has been found that some cervical ostium diameters are one (1) to two (2) millimeters at the entry point. In this embodiment, the cervix needs be dilated less than approximately three (3) millimeters in order for the distal four (4) millimeters of the facet of the device to enter the cervical cavity at the entry point with minimal bending of the abrasive material fibers. Flattening of the longitudinal axis of the device allows the slimmer version of the device to enter the cervix more evenly as the device is converted from a predominantly conical shape to a more ovoid shape much like a spatula with rounded edges.

In another embodiment of the invention, a device with maximum diameter three (3) millimeters and with abrasive material that is approximately 3.5 millimeters in length, if the distal four (4) millimeters of the head is inserted then the device tip including the abrasive material has a diameter at this point four (4) millimeters distal from the tip) of approximately 5.5 millimeters. In this embodiment, the cervix needs be dilated less than approximately 3.5 millimeters in order for the distal four (4) millimeters of the facet of the device to enter the cervical cavity.

In an embodiment of the invention, once the thin tapered device is inserted into the cervix, only the distal four (4) millimeters of the facet one (1) to two (2) square millimeters of the abrasive material need to be inside the canal to obtain sufficient material for a biopsy requiring fifteen 15 to fifty (50) copies of DNA. In an alternative embodiment of the invention, once the thin tapered device is inserted into the cervix, only the distal nine (9) millimeters of the facet corresponding to two (2) to four (4) square millimeters need to be inside the canal to obtain material for a biopsy requiring approximately 100 to 200 copies of DNA. In another embodiment of the invention, once the thin tapered device is inserted into the cervix, only the distal twelve (12) millimeters of the facet corresponding to four (4) to six (6) square millimeters need to be inside the canal to obtain material for a biopsy requiring approximately 300 to 500 copies of DNA. Unlike conventional curettage, the device can be rotated and the hooks will contact the os canal and frictionally abrade, circumferentially being pressed against the endocervical epithelium, while being pressed and rotated. Since the abrasive material and the spacing of the abrasive material combined have a greater propensity to ‘hold’ the tissue, more tissue is available for pathological analysis. This improves the diagnostic probability of determining the causative agent. Importantly, tissue yield is crucial when scanning pre-cancerous lesions.

### Inter Component Recordation

In another embodiment of the invention, a RFID tag is imbedded in one or more of: the head 120, the arm 782, the backstop 1190 the handle cannula or catheter 125, the catheter housing 355, a wrist bracelet worn by the physician, practitioner or examiner and optionally a base station. In an embodiment of the invention, the RFID tag is used to identify the head device and thereby determine the parameters under which the device was used. In one embodiment of the invention, the RFID tag operates using an Ultra High Frequency (UHF) signal. In another embodiment of the invention, the RFID tag operates using a microwave frequency signal.

In an embodiment of the present invention, a RFID reader is present in the operating theater which can then read, record and check the suitability of the components based on the RFID tags in the individual components. In an embodiment of the invention, the RFID reader can be positioned so that the RFID tag antenna is least affected by any conducting material. In one embodiment, the RFID tag is read-only. In another embodiment, the RFID tag contains an Electrically Erasable Programmable Read-Only Memory (EPROM), which enables both read and write functions. In an embodiment of the invention, the RFID tag is passive. In another embodiment of the invention, the RFID tag is semi-passive, containing a source of energy such as a battery to allow the tag to be constantly powered. In a further embodiment of the invention, the RFID tag is active, containing an internal power source, such as a battery, which is used to power any Integrated Circuits (ICs) in the tag and generate the outgoing signal. In another embodiment, the tag has the ability to enable location sensing through a photo sensor. In one embodiment of the invention, means of communication with a base station is embedded in one or more components. In one embodiment of the invention, the communication means utilizes one or more of a wireless local area network; a wireless wide area network; a cellular network; a satellite network; a Wi-Fi network; and a pager network. In one embodiment of the invention, a modem capable of communicating with one or more of the aforementioned networks is embedded in one or more components. In the following discussion the term ‘cellular modem’ will be used to describe the components embedded. The term ‘cellular modem’ will be herein used to identify any components of comparable size capable of communicating over one or more of the aforementioned networks. In one embodiment of the invention, the cellular modem can be a Code Division Multiple Access (CDMA) modem. In an embodiment of the invention, a RFID reader and associate integrated circuit processor can be embedded together with the cellular modem in one or more components. In such an embodiment, the RFID tags and RFID reader can be positioned to optimize the RFID read of the RFID tags from the available components.

### Surgical Procedures and Other Uses

In an embodiment of the invention, a system for using and monitoring a device during a surgical procedure comprises a head and handle, a comb for removing the tissue from the head, and a means for rotating the handle. The means for turning the head can include an automated device. The device can include an input module for selecting parameters for use in rotating the device, where the input module selects parameters based at least in part on the head device selected, a sensor for monitoring the head rotating velocity, a processor for comparing the rotational velocity of the head and the selected parameters and automatically adjusting the head rotation velocity when the comparison indicates an increased or decreased head rotation is required. The input module can receive audio, tactile or visual feedback to adjust the device during the surgical procedure.

In an embodiment of the invention, the device can be applied in any surgical, scientific, crime investigation or veterinary application that requires the use of a regulated constant or variable rotating tissue sampler. This can include laboratory equipment that requires tissue sampling, storage or any other clinical procedure.

### ASPECTS OF THE INVENTION

Some aspects of this invention include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, where the abrasive material is made up of a plurality of patches of abrasive material.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, where the un-expanded balloon at least partially fits within the cavity.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, where the balloon and the abrasive material is further adapted to at least partially fit within the cavity.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, where the balloon can be inflated to encompass a volume greater than the volume.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, where the abrasive material includes a plurality of fibers each with hooks to frictionally abrade the tissue.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, where insertion of the cannula inside a body canal and rotation of the cannula rotates the head inside the body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, where insertion of the cannula inside a body canal and rotation of the cannula rotates the head inside the body canal, where rotation of the cannula contacts the abrasive material with the epithelial tissue to obtain the biopsy tissue sample.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where one or both the head and the balloon are adapted to exit the catheter inside the body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where rotation of the cannula inside the catheter rotates the head inside the body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where rotation of the cannula inserted through the catheter rotates the balloon inside the body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where rotation of the cannula contacts the abrasive material with the epithelial tissue to obtain a biopsy tissue sample.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where the balloon can be deflated inside the body canal.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where the balloon can be deflated inside the body canal, where the balloon with the abrasive material can be reinserted into the catheter.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where the balloon can be deflated inside the body canal, where the balloon with the abrasive material can be reinserted into the cavity.

Further aspects include a device for contacting a tissue to obtain a biopsy sample comprising ahead with a proximal end, a distal end, a volume, and a cavity, where the proximal end of the head is attached to a cannula; a balloon adapted to expand from the cavity; and an abrasive material associated with the surface of the balloon, where the abrasive material on the balloon is adapted to contact the tissue to obtain the biopsy sample, further comprising a cannula and a lumen, where introduction of a gas or liquid through the lumen of the cannula expands the balloon, further comprising a catheter with a diameter, where the cannula and the head are adapted to be inserted into the catheter, where the catheter is adapted to be inserted into a body canal, where the balloon can be inflated to a size larger than the diameter of the catheter, where the balloon can be deflated inside the body canal, where the cannula with the balloon and the abrasive material can be withdrawn out of the catheter.

Some aspects of this invention include a method of obtaining a biopsy sample from epithelial tissue of a body canal comprising inserting a catheter into the body canal; inserting a cannula with a head including a balloon with abrasive material associated with the balloon into the catheter in the body canal; inflating the balloon inside the body canal, where the abrasive material contacts the epithelial tissue of the body canal; rotating the head in a first direction to frictionally abrade the epithelial tissue with the abrasive material; deflating the balloon; and removing the cannula with the head including the balloon and the abrasive material from the catheter.

Some aspects of this invention include a device for obtaining a biopsy tissue sample comprising a head with a proximal end, a distal end, and a diameter, where the proximal end of the head is attached to a cannula; a balloon associated with the head; an abrasive material associated with the surface of the balloon, where the balloon can be inflated; and abrasive material associated with the head.

Some aspects of this invention include a device for obtaining a biopsy tissue sample comprising a head with a proximal end, a distal end and a diameter, where the distal end is adapted to one or both introduce the device into the cervix and receive a handle to introduce the device into the cervix and the proximal end is raised in profile relative to the perimeter of the proximal end, where the surface of the proximal end is substantially covered with an abrasive material.

Some aspects of this invention include a device for obtaining a biopsy tissue sample comprising a head with a proximal end, a distal end and a first diameter, where the distal end is adapted to one or both introduce the device into the cervix and receive a handle to introduce the device into the cervix, where the surface of the proximal end is substantially covered with an abrasive material; and one or more blades with a proximal end, a distal end and a second diameter, where the one or more blades extend from the head, where the proximal end surface of one or more of the blades is substantially covered with an abrasive material.

Further aspects include a device for obtaining a biopsy tissue sample comprising a head with a proximal end, a distal end and a first diameter, where the distal end is adapted to one or both introduce the device into the cervix and receive a handle to introduce the device into the cervix, where the surface of the proximal end is substantially covered with an abrasive material; and one or more blades with a proximal end, a distal end and a second diameter, where the one or more blades extend from the head, where the proximal end surface of one or more of the blades is substantially covered with an abrasive material, where the proximal end of the head is raised in profile relative to the proximal end of one or more of the one or more blades.

Some aspects of this invention include a device for obtaining a biopsy tissue sample comprising a coil with a diameter between a lower limit of 1×10⁻³ m; and an upper limit of 1×10⁻² m, where the surface of the coil is substantially covered with an abrasive material.

Some aspects of this invention include a device for obtaining a biopsy tissue sample comprising a handle; a head attached to the handle with a facet and a railing substantially encircling the facet, where the railing has an abrasive edge; and an abrasive material adhered to the surface of the facet with an adhesive, where the adhesive is retained by the abrasive railing.

Some aspects of this invention include a device for contacting a tissue to obtain a biopsy sample comprising a catheter; a head with a proximal end, a distal end and a first diameter, where the proximal end of the head is attached to a cannula, where the head can be inserted into the catheter; an expandable balloon associated with the head; an abrasive material associated with the surface of the balloon, where the head can be pushed out of the catheter and the balloon expanded, where the abrasive material contacts the tissue to obtain the biopsy sample; and a backstop with a proximal end, a distal end associated with the head, where the backstop has abrasive material associated with the distal end, where the backstop has a second diameter greater than the first diameter, where the cannula is adapted to push the head and the backstop out of the catheter, where the cannula is adapted to allow the balloon to be expanded, where one or both the abrasive material on the balloon and the abrasive material associated with the backstop contacts the tissue to obtain the biopsy sample.

Some aspects of this invention include a kit for obtaining a biopsy tissue of epithelial tissue in a cavity comprising a catheter, where the catheter is adapted to be inserted into the cavity; and a device, including: a handle; a head attached to the handle with a facet and a backstop, where the head inserts into the catheter using the handle; and an abrasive material adhered to the surface of the facet and the surface of the backstop, where the handle pushes the head and the backstop out of the catheter to contact the epithelial tissue.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

The foregoing description of embodiments of the methods, systems, and components of the present invention has been provided for the purposes of illustration and description. It is not intended to be exhaustive or to limit the invention to the precise forms disclosed. Many modifications and variations will be apparent to one of ordinary skill in the relevant arts. For example, steps performed in the embodiments of the invention disclosed can be performed in alternate orders, certain steps can be omitted, and additional steps can be added. The embodiments were chosen and described in order to best explain the principles of the invention and its practical application, thereby enabling others skilled in the art to understand the invention for various embodiments and with various modifications that are suited to the particular used contemplated. Other embodiments are possible and are covered by the invention. Such embodiments will be apparent to persons skilled in the relevant art(s) based on the teachings contained herein. The breadth and scope of the present invention should not be limited by any of the above-described exemplary embodiments, but should be defined only in accordance with the following claims and their equivalents.

What is claimed is:

1. A device for obtaining epithelial tissue comprising:
(a) a shaft with a main axis longitudinal with the shaft and a pivot point at a shaft terminus;
(b) a first propeller blade with a proximal end and a distal end, where the first propeller blade tapers from the proximal end to a propeller tip at the distal end, where the first propeller blade is attached to the shaft and adapted to substantially rotate 360 degrees about the pivot point, where the first propeller blade includes a first facet, where the first facet has a first rail around the perimeter; and
(c) a first abrasive material substantially covering the first facet, where the first abrasive material is adapted to contact a first epithelium to obtain a first epithelial tissue, where the first propeller blade includes a first smooth backside.

2. The device of claim 1, where the shaft is adapted to be held between an index finger and a thumb while in contact with the first epithelium to obtain the first epithelial tissue in the first abrasive material.

3. The device of claim 1, where insertion of the propeller tip with the smooth backside into an orifice dilates the orifice thereby contacting the first abrasive material onto the first epithelium.

4. The device of claim 1, where the first abrasive material includes a plurality of fibers each with hooks to frictionally abrade the first epithelium.

5. The device of claim 1, where an adhesive affixes the first abrasive material to the first facet.

6. The device of claim 5, where the first rail forms a dam which confines the spread of the adhesive.

7. The device of claim 1, where the first propeller blade can be rotated about the pivot point to allow the first propeller blade to be positioned substantially parallel to the main axis of the shaft.

8. The device of claim 1, where the first propeller blade can be rotated about the pivot point to allow the first propeller blade to be positioned substantially perpendicular to the main axis of the shaft.

9. The device of claim 1, where rotation of the shaft around the main axis contacts the first abrasive material with the first epithelium to obtain a first epithelial tissue.

10. The device of claim 1, further comprising:
(d) a second propeller blade, where the second propeller blade is adapted to substantially rotate 360 degrees about the pivot point.

11. The device of claim 10, where the second propeller blade can be rotated about the pivot point to allow the second propeller blade to be positioned substantially parallel to the main axis of the shaft.

12. The device of claim 10, where the second propeller blade can be rotated about the pivot point to allow the second propeller blade to be positioned substantially perpendicular to the main axis of the shaft.

13. The device of claim 10, where the second propeller blade includes a second facet substantial covered with a second abrasive material.

14. The device of claim 10, where the second propeller blade includes a second smooth backside.

15. The device of claim 14, where the first propeller blade and the second propeller blade can be positioned such that the first abrasive material and the second smooth backside are distal to the shaft terminus.

16. A method of obtaining epithelial tissue comprising:
(a) receiving a shaft with a main axis of rotation longitudinal with the shaft and a first propeller blade attached to the shaft at a pivot point located at a shaft terminus, where the first propeller blade has a proximal end and a distal end, where the first propeller blade tapers from the proximal end to a propeller tip at the distal end, where the first propeller blade includes a first facet and a first abrasive material substantially covering the first facet;
(b) orienting the first propeller blade parallel with the shaft, where the first propeller blade includes a first abrasive material and a first smooth backside, where the first propeller blade is rotated about the pivot point to orient the first propeller blade parallel with the shaft;
(c) inserting the first propeller blade into an orifice to dilate the orifice, where after dilation the first propeller blade contacts the first abrasive material onto a first epithelium;
(d) rotating the shaft to abrade the first epithelium with the first abrasive material; and
(e) removing the shaft with the first propeller blade and a first epithelial tissue contained in the first abrasive material.

17. The method of claim 16, further comprising:
(f) receiving a second propeller blade attached to the shaft at the pivot point, where the second propeller blade includes a second abrasive material and a second smooth backside;
(g) adjusting the second propeller blade substantially perpendicular to the main axis of rotation, where the second abrasive material is distal to the shaft terminus;
(i) where rotating the shaft in step (d), frictionally abrades a second epithelium with the second abrasive material; and
(j) where removing the shaft in step (e), removes the first propeller blade and a first epithelial tissue contained in the first abrasive material and the second propeller blade with a second epithelial tissue contained in the second abrasive material.

18. The method of claim 16, further comprising:
(f) receiving a second propeller blade attached to the shaft at the pivot point, where the second propeller blade includes a second smooth backside; and
(g) adjusting the second propeller blade substantially perpendicular to the main axis of rotation, where the second smooth backside is distal to the shaft terminus.

19. The method of claim 16, further comprising:
(f) adjusting the first propeller blade substantially perpendicular to the main axis of rotation, where the first abrasive material is distal to the shaft terminus;
(g) receiving a second propeller blade attached onto the shaft at the pivot point, where the second propeller blade includes a second smooth backside; and
(h) adjusting the second propeller blade substantially perpendicular to the main axis of rotation, where the second smooth backside is distal to the shaft terminus.

20. A kit for obtaining epithelial tissue comprising:
(a) a shaft with a proximal end, a distal end, a main axis of rotation longitudinal with the shaft, and a pivot point where the pivot point is located toward the distal end;
(b) a propeller blade attachable to the shaft at the pivot point and adapted to rotate about the pivot point to a number of positions including recessed, perpendicular to the main axis of rotation, 30 degrees to the main axis of rotation and parallel with the main axis of rotation, where the propeller blade includes an abrasive material adhered to a facet on the propeller blade, where the propeller blade tapers from the proximal end to a propeller tip at the distal end, where the propeller blade includes a smooth backside; and
(c) instructions for attaching the propeller blade to the shaft, adjusting the propeller blade relative to the shaft in a first position, inserting the propeller tip into a cervix, contacting the abrasive material on epithelium, turning the shaft to obtain an epithelial tissue from the epithelium with the abrasive material and removing the shaft, the propeller blade and the epithelial tissue contained in the abrasive material.
